(19) United States
(12) Patent Application Publication
(10) Pub. No.: US 2026/0215786 A1
(43) Pub. Date: Jul. 30, 2026

(54) CLIP INSTRUMENTS

(71) Applicant: HANGZHOU AGS MEDTECH CO., LTD., Hangzhou (CN)

(72) Inventors: Feng YAO, Hangzhou (CN); Zhongli ZHENG, Hangzhou (CN); Guoqing WANG, Hangzhou (CN); Chunqi HAN, Hangzhou (CN); Jianyang FENG, Hangzhou (CN)

(73) Assignee: HANGZHOU AGS MEDTECH CO., LTD., Hangzhou (CN)

(21) Appl. No.: 19/457,301

(22) Filed: Jan. 23, 2026

Related U.S. Application Data
(60) Provisional application No. 63/754,009, filed on Feb. 5, 2025.

(30) Foreign Application Priority Data
Jan. 25, 2025 (CN) 202510121172.2
Jan. 9, 2026 (CN) 202610030880.X

(51) Int. Cl. A61B 17/128; A61B 17/122; A61B 17/00

(52) U.S. Cl. CPC A61B 17/1285; A61B 17/1227; A61B 2017/00473

(57) ABSTRACT

Embodiments of the present disclosure provide a clip instrument. The clip instrument may include a tube body; a clip, comprising at least two clamping components; a locking component, releasably connected with the tube body and capable of locking the at least two clamping components; a connection component, slidably connected with the tube body along a first direction and capable of moving relative to the locking component along the first direction.

### CROSS-REFERENCE TO RELATED APPLICATIONS

This application claims priority of Chinese Application No. 202510121172.2 filed on January 25, 2025, US Provisional Application No. US 63/754,009 filed on February 5, 2025, and Chinese Application No. 202610030880.X filed on January 9, 2026, the contents of each of which are hereby incorporated by reference in its entirety.

### TECHNICAL FIELD

The present disclosure relates to the field of medical instruments, and in particular, to clip instruments.

### BACKGROUND

The stomach or intestines of living organisms often experience bleeding, mucosal injury, or even perforation due to various diseases, accidental injuries, or injuries during endoscopic diagnosis or therapeutic procedures. Clinically, a clip instrument may be used for mechanical compression hemostasis, and the clip instrument may grasp the tissue around a wound and temporarily hold wound edges together to achieve closure. The clip instrument is also applied in wound suturing. Current clip instruments have a relatively long length of clip blades that remains retained within the organism. How to reduce the length of the clip blades retained in the organism is a problem that needs to be addressed.

Therefore, it is necessary to provide an improved clip instrument.

### SUMMARY

One or more embodiments of the present disclosure provide a clip instrument. The clip instrument may include a tube body. A clip, comprising at least two clamping components. A locking component, releasably connected with the tube body and capable of locking the at least two clamping components. A connection component, slidably connected with the tube body along a first direction and capable of moving relative to the locking component along the first direction.

One or more embodiments of the present disclosure provide a clip instrument. The clip instrument may include a tube body. A clip, comprising at least two clamping components and a connecting portion, the connecting portion includes at least two connecting arms extending toward a distal end, and each of the at least two connecting arms is releasably connected with one of the at least two clamping components. A locking component, releasably connected with the tube body and capable of locking the at least two clamping components

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an exemplary clip instrument according to some embodiments of the present disclosure;

FIG. 2 is a schematic diagram illustrating a cross-sectional view along line A-A of the clip instrument shown in FIG. 1 according to some embodiments of the present disclosure;

FIG. 3 is a schematic diagram illustrating an exemplary locking component according to some embodiments of the present disclosure;

FIG. 4A is a schematic diagram illustrating an exemplary clamping component according to some embodiments of the present disclosure;

FIG. 4B is a schematic diagram illustrating an exemplary clamping component according to some other embodiments of the present disclosure;

FIG. 5 is a schematic diagram illustrating an exemplary connecting block according to some embodiments of the present disclosure;

FIG. 6 is a schematic diagram illustrating an installation process of a connecting block according to some embodiments of the present disclosure;

FIG. 7 is a schematic diagram illustrating cross-sectional views along lines A-A and B-B of FIG. 6 according to some embodiments of the present disclosure;

FIG. 8 is a schematic diagram illustrating an installation process of a connecting block according to some other embodiments of the present disclosure;

FIG. 9 is a schematic diagram illustrating an exemplary tube body according to some embodiments of the present disclosure;

FIG. 10 is a schematic diagram illustrating a disassembly process of a locking component and a tube body according to some embodiments of the present disclosure;

FIG. 11 is a schematic diagram illustrating a disassembly process of a locking component and a tube body according to some other embodiments of the present disclosure;

FIG. 12 is a schematic diagram illustrating an exemplary connecting block according to some other embodiments of the present disclosure;

FIG. 13 is a schematic diagram illustrating an installation process of a connecting block according to some other embodiments of the present disclosure;

FIG. 14 is a schematic diagram illustrating an exemplary tube body according to some other embodiments of the present disclosure;

FIG. 15 is a schematic diagram illustrating an exemplary locking component according to some other embodiments of the present disclosure;

FIG. 16 is a schematic diagram illustrating an exemplary outer tube according to some other embodiments of the present disclosure;

FIG. 17 is a schematic diagram illustrating an installation process of a connecting block according to some other embodiments of the present disclosure;

FIG. 18 is a schematic diagram illustrating an exemplary connecting block according to some other embodiments of the present disclosure;

FIG. 19A is a schematic diagram illustrating an exemplary locking component according to some other embodiments of the present disclosure;

FIG. 19B is a schematic diagram illustrating an exemplary locking component according to some other embodiments of the present disclosure;

FIG. 20 is a schematic diagram illustrating a disassembly process of a locking component and an outer tube according to some other embodiments of the present disclosure;

FIG. 21 is a schematic diagram illustrating an exemplary connection component according to some embodiments of the present disclosure;

FIG. 22A is a schematic diagram illustrating connection between two clamping components and a connection component according to some embodiments of the present disclosure;

FIG. 22B is a schematic diagram illustrating a cross-sectional view of a connection between two clamping components and a connection component according to some other embodiments of the present disclosure;

FIG. 23 is a schematic diagram illustrating a disassembly process of two clamping components and a connection component according to some embodiments of the present disclosure;

FIG. 24 is a schematic diagram illustrating a disassembly process of two clamping components and a connection component according to some embodiments of the present disclosure;

FIG. 25A is a schematic diagram illustrating a locking process of two clamping components and a locking component according to some embodiments of the present disclosure;

FIG. 25B is a schematic diagram illustrating a sectional view of FIG. 25A according to some embodiments of the present disclosure;

FIG. 26 is a schematic diagram illustrating an exemplary clamping component according to some other embodiments of the present disclosure;

FIG. 27 is a schematic diagram illustrating an exemplary connection component according to some other embodiments of the present disclosure;

FIG. 28 is a schematic diagram illustrating an exemplary tube body according to some other embodiments of the present disclosure;

FIG. 29 is a schematic diagram illustrating a closed state of a clamping component according to some other embodiments of the present disclosure;

FIG. 30 is a schematic diagram illustrating a locking process of connection component and a locking component according to some other embodiments of the present disclosure;

FIG. 31A is a schematic diagram illustrating an exemplary connection component according to some other embodiments of the present disclosure;

FIG. 31B is a schematic diagram illustrating connection of a connection component according to some other embodiments of the present disclosure;

FIG. 32 is a schematic diagram illustrating an exemplary connection component according to some other embodiments of the present disclosure;

FIG. 33 is a schematic diagram illustrating connection of a connection component according to some other embodiments of the present disclosure;

FIG. 34 is a schematic diagram illustrating a locking process of two clamping components according to some other embodiments of the present disclosure;

FIG. 35 is a schematic diagram illustrating a disassembly process of two clamping components and a connection component according to some other embodiments of the present disclosure; and

FIG. 36 is a schematic diagram illustrating a clip instrument according to some embodiments of the present disclosure.

### DETAILED DESCRIPTION

To illustrate the technical solutions in the embodiments of the present disclosure more clearly, the following briefly introduces the accompanying drawings required for describing the embodiments. Obviously, the accompanying drawings in the following description are only some examples or embodiments of the present disclosure. For a person of ordinary skill in the art, the present disclosure may be applied to other similar scenarios based on these drawings without creative efforts. Unless obviously obtained from the context or the context illustrates otherwise, the same numeral in the drawings refers to the same structure or operation.

As shown in the present disclosure and the claims, unless the context clearly indicates an exception, the terms "a," "an," "one," and/or "the" are not intended to refer specifically to a singular form, and may also include a plural form. Generally, the terms "include" and "contain" only indicate inclusion of explicitly identified steps and elements. These steps and elements do not constitute an exclusive list. A manner or device may also include other steps or elements.

It should be noted that the terms "distal end" and "proximal end" in the present disclosure are configured to describe relative positions of components of a medical instrument. The "proximal end" refers to an end of a medical instrument component that is closer to an operator of the instrument or farther from a treatment object during a medical operation. The "distal end" refers to an end of a medical instrument component that is farther from the operator of the instrument or closer to the treatment object during the medical operation.

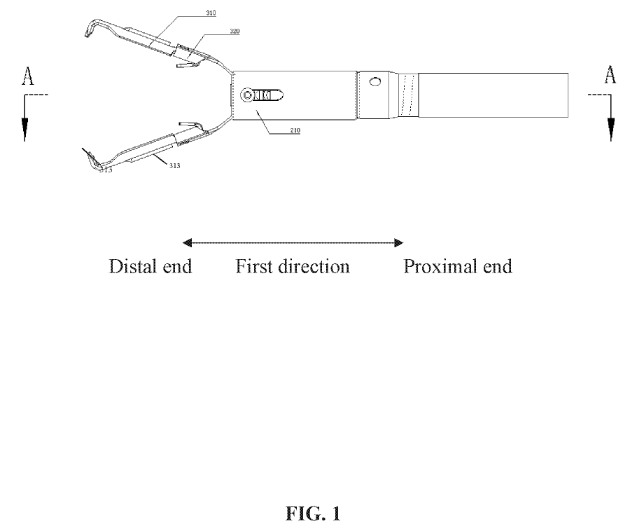

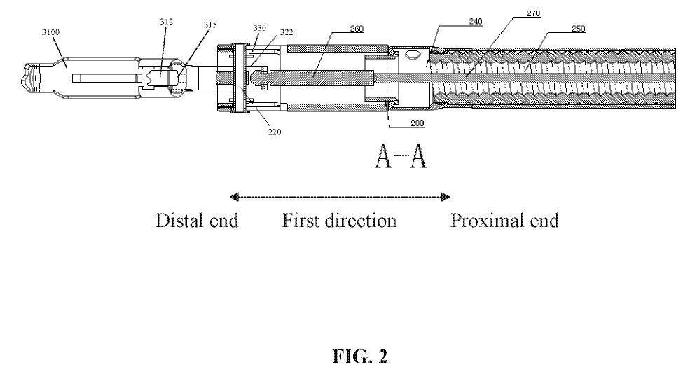

FIG. 1 is a schematic diagram illustrating an exemplary clip instrument according to some embodiments of the present disclosure. FIG. 2 is a schematic diagram illustrating a cross-sectional view along line A-A of a clip instrument shown in FIG. 1 according to some embodiments of the present disclosure.

Some embodiments of the present disclosure provide a clip instrument. As shown in FIG. 1 and FIG. 2, the clip instrument includes a tube body 210, a clip, a locking component 330 and a connection component. The clip includes at least two clamping components 310. The locking component 330 is releasably connected with the tube body 210 and capable of locking the at least two clamping components 310. The connection component is slidably connected with the tube body 210 along a first direction and capable of moving relative to the locking component 330 along the first direction.

The tube body 210 is configured to provide an accommodating space for at least a portion of each of one or more components (e.g., the locking component 330 and the connection component) of the clip instrument. The tube body 210 may be set as a hollow tube. The tube body 210 may be made of a material that balances biocompatibility, mechanical strength, flexibility, corrosion resistance, or the like. Exemplary materials of the tube body 210 may include at least one of polytetrafluoroethylene, polyamide, polyethylene, polyurethane, etc.

The clip is configured to implement a clamping function. The distal ends of the at least two clamping components 310 may be aligned and abut against each other to implement clamping. For example, the distal ends of two clamping components 310 are bent toward the inner side of each of the two clamping components 310, so that opposing inner-facing side surfaces of the distal ends of the two clamping components 310 may be aligned and abut each other. As another example, each of the distal ends of the three clamping components 310 is bent toward the inner side of each of the three clamping components 310, so that opposing inner-facing side surfaces of the distal ends of the three clamping components 310 may be aligned and abut each other. An inner side refers to a side that is closer to the central axis of the tube body 210 along a radial direction of the tube body 210. An outer side refers to a side that is away from the central axis of the tube body 210 along the radial direction of the tube body 210. Merely by way of example, the clip may open to grasp tissue around a wound of a living body through the two clamping components 310 and then close the two clamping components 310 to hold edges of the wound together, thereby closing the wound.

In some embodiments, each of the at least two clamping components 310 may be sheet-shaped and bent at the distal ends of the at least two clamping components 310, so that a distal end of at least one of the at least two clamping components 310 may be aligned with and abut against a distal end of other one of the at least two clamping component 310. The material of one of the at least two clamping components 310 may be stainless steel, titanium alloy, or the like, which balances biocompatibility, mechanical strength, corrosion resistance, etc.

The locking component 330 is configured to provide a closing constraint for the two clamping components 310. The locking component 330 is configured to provide a closing constraint for the at least two clamping components 310, i.e., to keep the at least two clamping components 310 in a closed state. In some embodiments, the locking component 330 is releasably connected to the tube body 210 and is capable of locking the at least two clamping components 310. A releasable connection refers to that the connection between two connected parts (e.g., the locking component 330 and the tube body 210) may be disengaged. During use of the clip instrument the two parts that is in a releasable connection may be connected or separated as required. Merely by way of example, releasable connections include snap-fit, magnetic attraction, plug-in, and threaded engagement, etc.

The connection component is configured to connect the tube body 210 with the locking component 330. In some embodiments, the connection component is slidably connected with the tube body 210 along a first direction. The first direction refers to an extension direction of the central axis of the tube body 210. In some embodiments, the connection component is capable of moving relative to the locking component 330 along the first direction, thereby controlling connection and disconnection between the locking component 330 and the tube body 210. More descriptions may be found in below.

In some embodiments of the present disclosure, the tube body 210 and the locking component 330 are configured to be separable, so that the stroke required for opening and closing the at least two clamping components 310 may be accommodated within the tube body 210, thereby shortening the length of the locking component 330 and significantly reducing the portion of the clip instrument that remains inside the body, which improves adaptability.

In some embodiments, as shown in FIG. 2, the locking component 330 includes a connecting channel, and the connection component includes a first limiting shaft 220.

The connecting channel is used for the connection between the first limiting shaft 220 and the locking component 330. For example, the connecting channel may limit the first limit shaft 220 within the connecting channel to keep relative position between the first limit shaft 220 and the locking component 330 unchanged.

In some embodiments, the first limiting shaft 220 is columnar and extends through the tube body 210 and the locking component 330 in a direction perpendicular to the first direction, and is movable along the first direction.

In some embodiments, when the connection component is in a first position, the first limiting shaft 220 is located within the connecting channel, thereby connecting the tube body 210 and the locking component 330. When the connection component is in a second position, the first limiting shaft 220 is disengaged from the connecting channel, so that the tube body 210 and the locking component 330 are no longer connected and may be separated from each other. The first position refers to a position of the connection component when the first limiting shaft 220 is within the connecting channel, and the second position refers to a position when the first limiting shaft 220 is outside the connecting channel. The first position and the second position may be set as required, and the first position and the second position may be represented by distance from a distal end (or a proximal end) of the connection component to the distal end of the tube body 210. The connecting channel may be a slot structure or a hole structure. When the connecting channel is a through slot structure, the first limiting shaft 220 may be disengaged from a through opening of the slot structure. When the connecting channel is a closed hole structure, the first limiting shaft 220 may be disengaged from the hole by deforming the hole structure or by disengaging from contact with the hole structure.

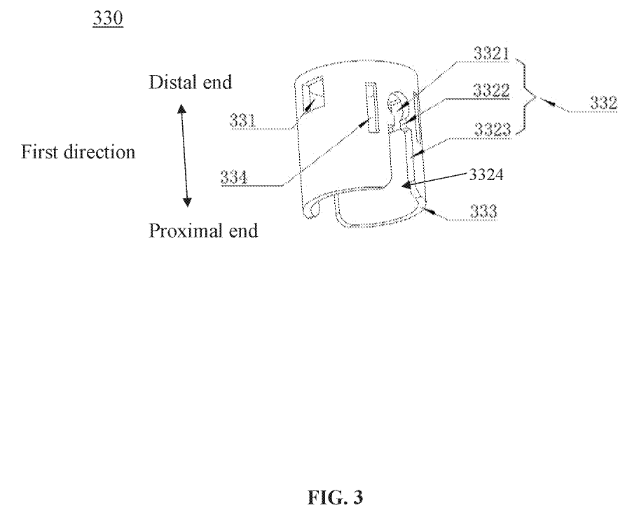

FIG. 3 is a schematic diagram illustrating an exemplary locking component according to some embodiments of the present disclosure.

In some embodiments, as shown in FIG. 3, the locking component 330 is provided with a guiding groove 332, the guiding groove 332 includes a first groove portion 3321 and a second groove portion 3323, the second groove portion 3323 is located at a proximal end of the first groove portion 3321, and a first limiting structure 3322 is provided between the second groove portion 3323 and the first groove portion 3321, and the first groove portion 3321 constitutes the connecting channel.

The first limiting structure 3322 is a structure protruding toward an interior of the guide groove 332. When the first limiting shaft 220 is located in the first groove portion 3321, a closed end at the distal end of the guide groove 332 and the first limiting structure 3322 limit the first limiting shaft 220 to fix a relative position between the first limiting shaft 220 and the locking component 330.

In some embodiments, after the first limiting shaft 220 moves from the distal end of the clip instrument toward the proximal end of the clip instrument and passes the first limiting structure 3322 from the first groove portion 3321, the connection component may move to arrive at the second position. The first limiting shaft 220 passing the first limiting structure 3322 may refer to a structure of the first limiting structure 3322 being damaged by the first limiting shaft 220 under the external force, causing the first limiting structure 3322 to curl inward or outward, or causing the first limiting structure 3322 to be squeezed and deformed to become smooth and no longer have a limiting function.

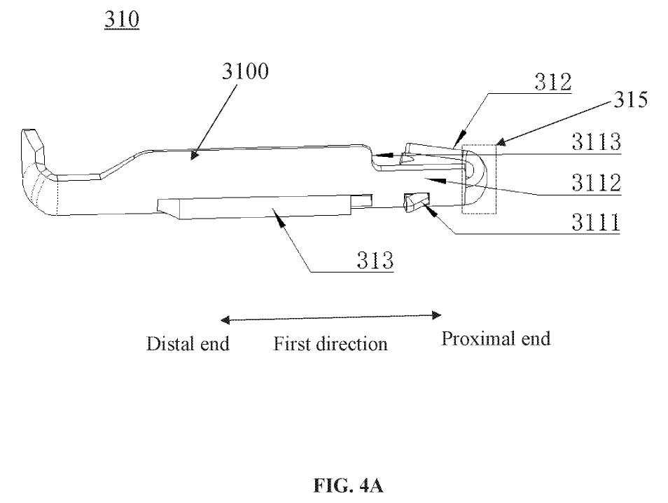

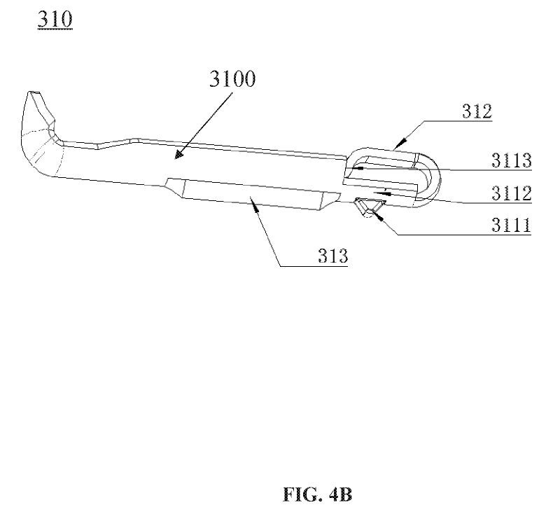

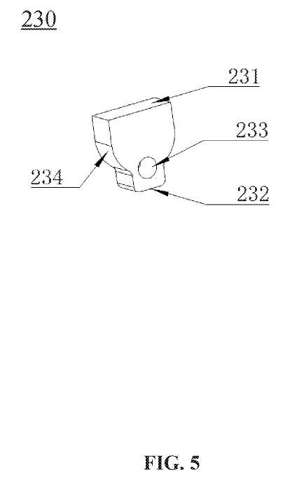

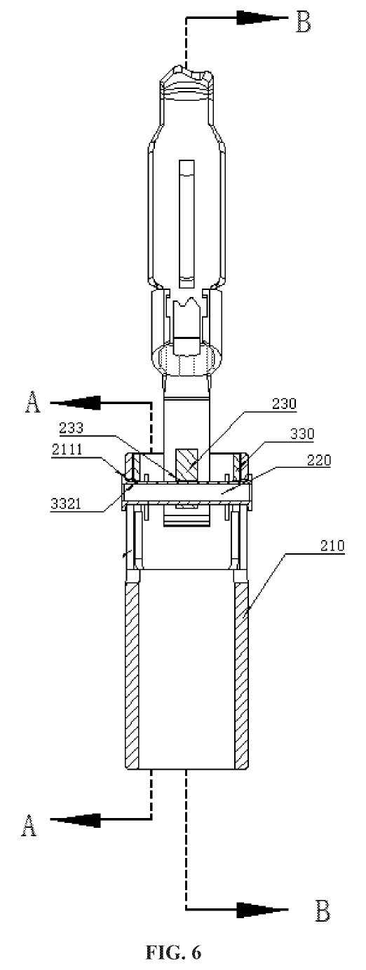

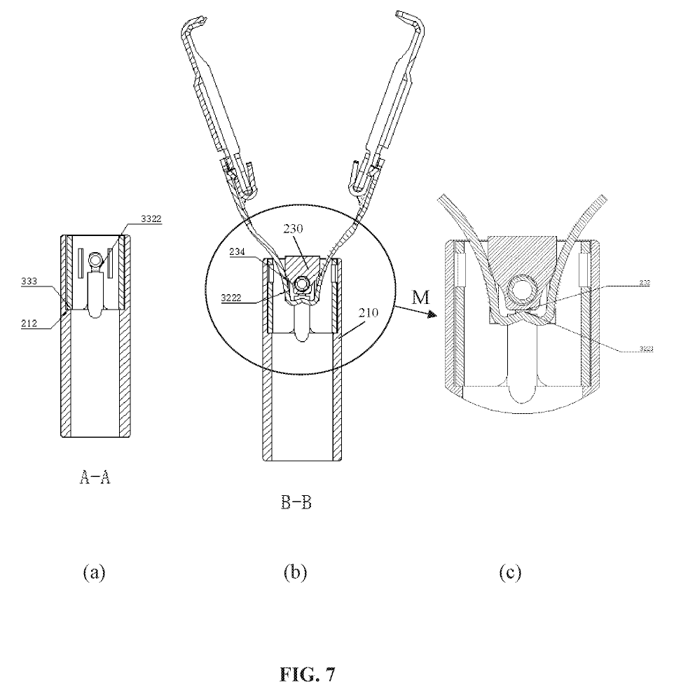

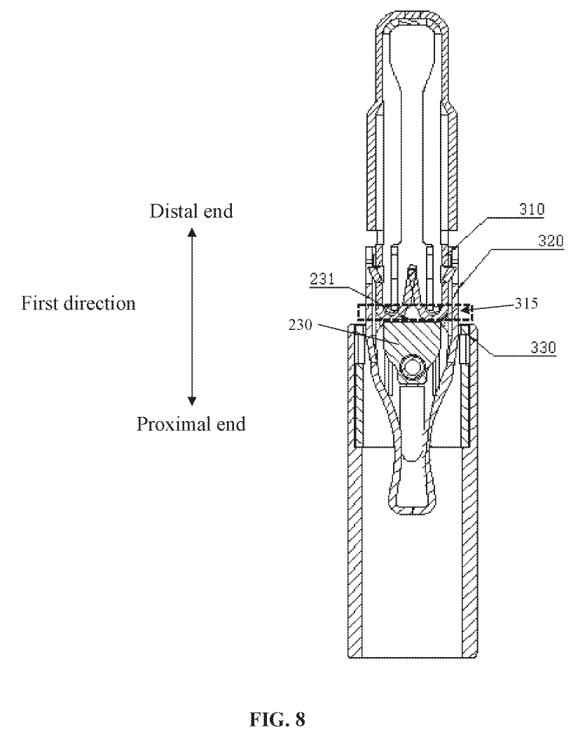

FIG. 4A is a schematic diagram illustrating an exemplary clamping component according to some embodiments of the present disclosure. FIG. 4B is a schematic diagram illustrating an exemplary clamping component according to some other embodiments of the present disclosure. FIG. 5 is a schematic diagram illustrating an exemplary connecting block according to some embodiments of the present disclosure. FIG. 6 is a schematic diagram illustrating an installation process of a connecting block according to some embodiments of the present disclosure. FIG. 7 is a schematic diagram illustrating cross-sectional views along lines A-A and B-B of FIG. 6 according to some embodiments of the present disclosure, wherein (a) is a cross-sectional view taken along A-A in FIG. 6, (b) is a cross-sectional view taken along B-B in FIG. 6, and (c) is an enlarged view of the M section in (b). FIG. 8 is a schematic diagram illustrating an installation process of a connecting block according to some other embodiments of the present disclosure.

In some embodiments, as shown in FIG. 4A and FIG. 4B, the clip includes an abutting portion 315, and movement of the abutting portion 315 toward a proximal end of the clip instrument drives the connection component to move toward the proximal end of the clip instrument.

The abutting portion 315 may be located at a proximal end of the clip. The abutting portion 315 may be a straight plate structure or an arc-plate structure connected to the proximal end of the clip. When the abutting portion 315 is the arc-plate structure, the abutting portion 315 may present an arcuate surface that protrudes toward the proximal end of the abutting portion 315 to facilitate abutment with the connection component. In some embodiments, the abutting portion 315 abuts against the first limiting shaft 220 and drives the first limiting shaft 220 to move, causing the first limiting shaft 220 to travel from the distal end of the clip instrument toward the proximal end of the clip instrument and pass the first limiting structure 3322 from the first groove portion 3321.

In some embodiments, as shown in FIG. 5, FIG. 6, FIG. 7 and FIG. 8, the connection component includes a connecting block 230. The first limiting shaft 220 is connected with the connecting block 230. After the abutting portion 315 moves to the proximal end of the clip instrument until the abutting portion 315 abuts the connecting block 230, the abutting portion 315 drives the connection component to move to the proximal end of the clip instrument.

In some embodiments, the connecting block 230 passes through the first limiting shaft 220. For example, the connecting block 230 is provided with a first shaft hole 233, and the first limiting shaft 220 passes through the first shaft hole 233 to connect with the connecting block 230.

In some embodiments, the abutting portion 315 abuts against the connecting block 230 and drives the first limiting shaft 220 to move via the connecting block 230, causing the first limiting shaft 220 to travel from the distal end of the clip instrument toward the proximal end of the clip instrument and pass the first limiting structure 3322 to exit the first groove portion 3321.

In some embodiments, when the abutting portion 315 moves to the proximal end of the clip instrument until the abutting portion 315 abuts against the connecting block 230, a distal end 231 of the connecting block 230 may be closer to the distal end of the clip instrument than the distal ends of the tube body 210 and the locking component 330. In other words, the distal end 231 of the connecting block 230 protrudes a little beyond the tube body 210 and the distal end of the locking component 330, thereby ensuring that at that moment, the at least two clamping component 310 is out of contact with both the tube body 210 and the locking component 330, thereby improving tactile feedback and operational smoothness.

In some embodiments, the locking component 330 is a locking tube having a hollow tubular configuration. The guiding groove 332 is provided at a proximal end 333 of the locking tube, and the guiding groove 332 has an opening facing the proximal end 333 of the locking tube.

In some embodiments, after the first limiting shaft 220 passes the first limiting structure 3322, the first limiting shaft 220 may first pass through the second groove portion 3323 and then be disengaged from the opening 3324. The buffering and guiding functions provided by the second groove portion 3323 enhance the stability and applicability of the clip instrument.

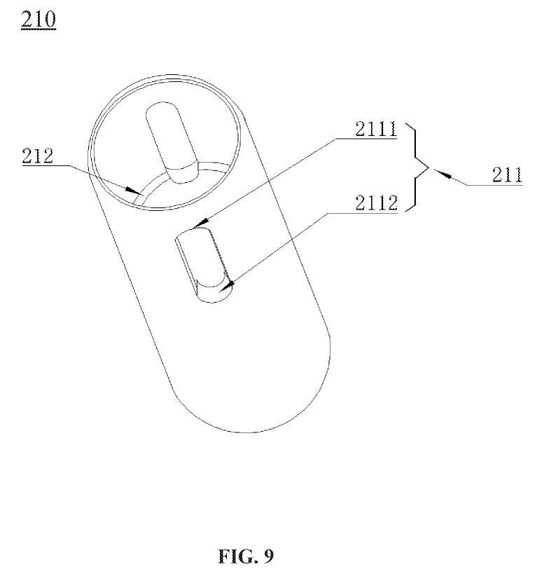

FIG. 9 is a schematic diagram illustrating an exemplary tube body according to some embodiments of the present disclosure.

In some embodiments, as shown in FIG. 2 and FIG. 9, at least a portion of the locking tube is located inside the tube body 210, the tube body 210 includes a sliding groove 211, and the guiding groove 332 at least partially overlaps with the sliding groove 211. The first limiting shaft 220 may simultaneously extend through both the guide groove 332 and the sliding groove 211, thereby connecting the locking component 330 to the tube body 210.

In some embodiments, the two sliding grooves 211 may be oppositely arranged on a side wall of the tube body 210, i.e., the two sliding grooves 211 may be symmetrical relative to a first axial section passing through the central axis of the tube body 210 and the two sliding grooves 211 may be located on a second axial section passing through the central axis of the tube body 210. The first axial section is perpendicular to the second axial section. The two guide grooves 332 may be oppositely arranged on a side wall of the locking component 330, i.e., the two guide grooves 332 may be symmetrical relative to a third axial section passing through the central axis of the tube body 210 and the two guide grooves 332 are located on a fourth axial section passing through the central axis of the tube body 210. The third axial section is perpendicular to the fourth axial section. The first limiting shaft 220 may simultaneously pass through the two sliding grooves 211 and the two guide grooves 332, i.e., the two sliding grooves 211 and the two guide grooves 332 are aligned. The second axial section where the two sliding grooves 211 are located and the fourth axial section where the two guide grooves 332 are located are the same axial section. The first limiting shaft 220 is located on the same axial section.

In some embodiments, the first limiting shaft 220 may move relative to the tube body 210, i.e., the first limiting shaft 220 may move between a distal end 2111 of the sliding groove 211 and a proximal end 2112 of the sliding groove 211.

In some embodiments, as the first limiting shaft 220 moves within the sliding groove 211 from the distal end 2111 of the sliding groove 211 toward the proximal end 2112 of the sliding groove 211, the first limiting shaft 220 may pass the first limiting structure 3322 to be disengage from the first groove portion 3321, pass through the second groove portion 3323, and then separate from the guide groove 332 via the opening 3324, thereby disconnecting the first limiting shaft 220 from the locking component 330 and, in turn, releasing the connection between the locking component 330 and the tube body 210.

In some embodiments, a limiting portion 212 is provided inside the tube body 210, and the proximal end 333 of the locking tube abuts against the limiting portion 212 to restrict movement of the locking tube toward the proximal end of the tube body 210, thereby preventing the locking tube from moving proximally together with the first limiting shaft 220.

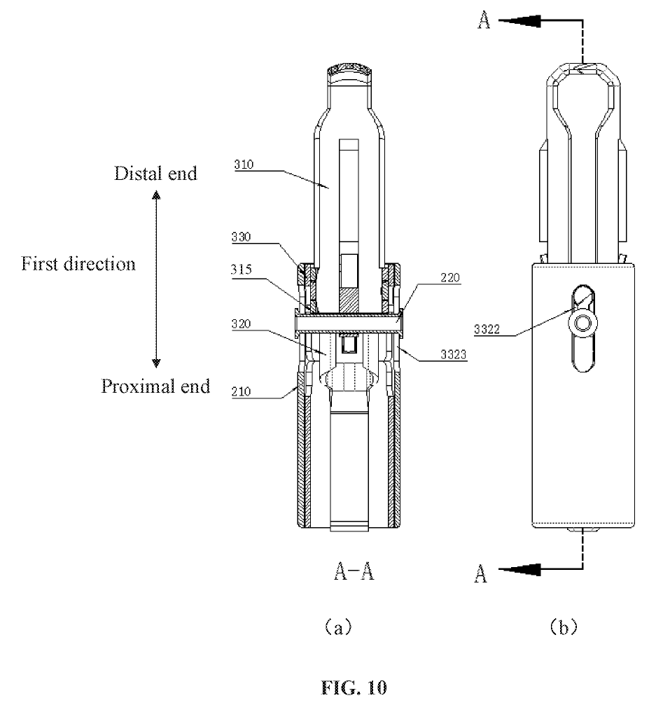

FIG. 10 is a schematic diagram illustrating a disassembly process of a locking component and a tube body according to some embodiments of the present disclosure, wherein (a) is a cross-sectional view along A-A in image (b)

Merely by way of example, reference to FIG. 10, when the at least two clamping components 310 are in the closed state, the clip moves from the distal end of the clip instrument toward the proximal end of the clip instrument until the abutting portion 315 abuts against the distal end 231 of the connecting block 230. The clip drives the first limiting shaft 220 to move to the proximal end of the clip instrument and pass the first limiting structure 3322 of the locking component 330, i.e., to be disengaged from the first groove portion 3321 and enter the second groove portion 3323, thereby releasing the connection between the locking component 330 and the tube body 210 and achieving separation of the locking component 330 from the tube body 210.

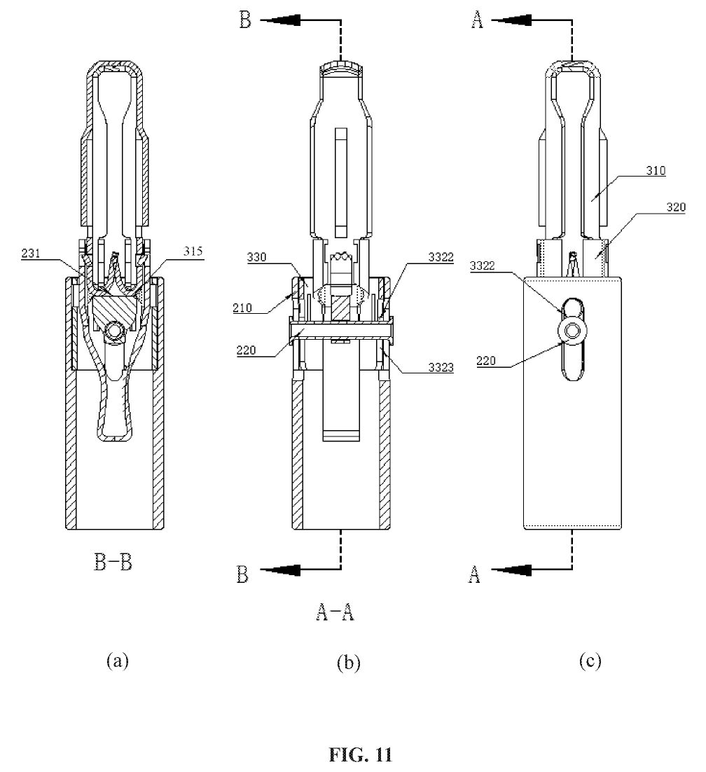

FIG. 11 is a schematic diagram illustrating a disassembly process of a locking component and a tube body according to some other embodiments of the present disclosure, wherein (b) is a cross-sectional view along A-A in image (c), and (a) is a cross-sectional view along B-B in image (b).

Merely by way of example, reference to FIG. 11, when the at least two clamping components 310 are in the closed state, the connecting portion 320 continues to drive the clamping component 310 to move toward the proximal end of the clip instrument. The abutting portion 315 presses against the first limiting shaft 220, causing the first limiting shaft 220 to move toward the proximal of the tube body 210 end until the first limiting shaft 220 passes the first limiting structure 3322 of the locking component 330 and enters the second groove portion 3323, thereby releasing the connection between the locking component 330 and the tube body 210 and achieving separation of the locking component 330 from the tube body 210.

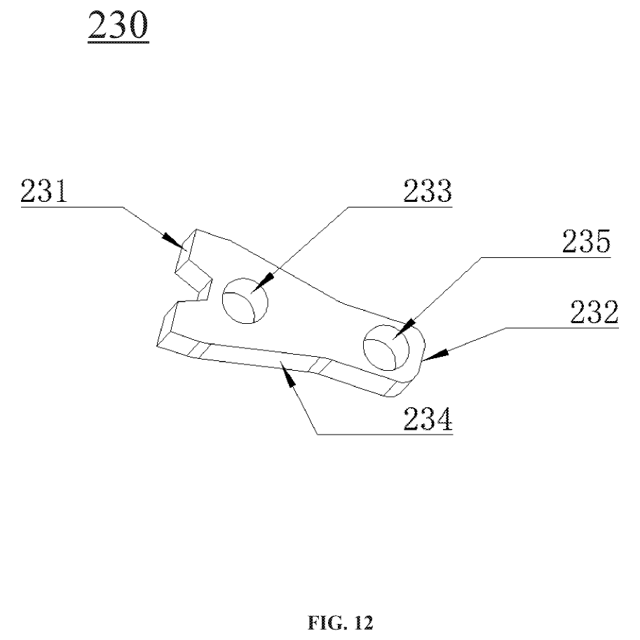

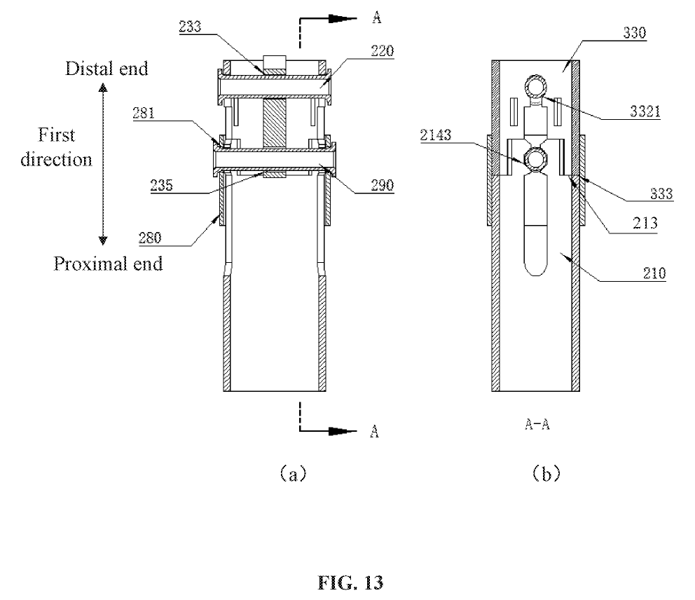

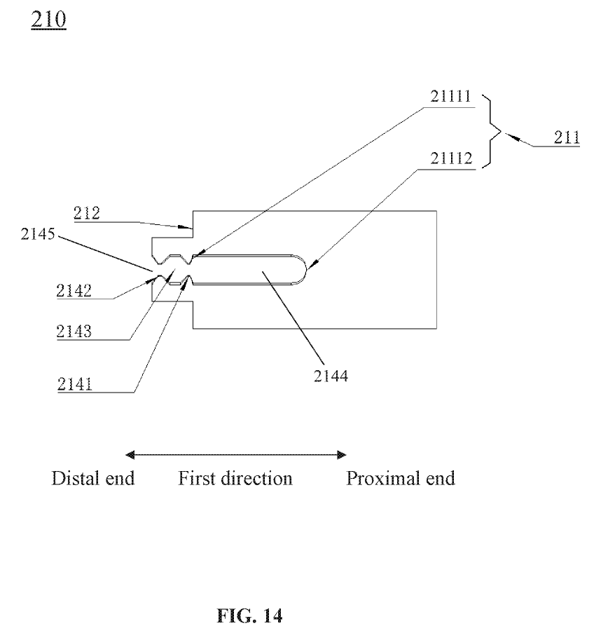

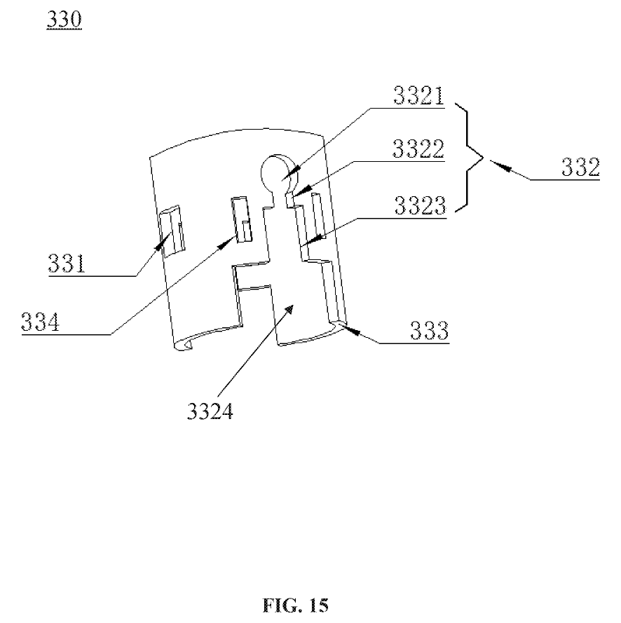

FIG. 12 is a schematic diagram illustrating an exemplary connecting block according to some other embodiments of the present disclosure. FIG. 13 is a schematic diagram illustrating an installation process of a connecting block according to some other embodiments of the present disclosure, wherein (b) is a cross-sectional view along A-A in image (a). FIG. 14 is a schematic diagram illustrating an exemplary tube body according to some other embodiments of the present disclosure. FIG. 15 is a schematic diagram illustrating an exemplary locking component according to some other embodiments of the present disclosure.

In some embodiments, and as shown in FIG. 12, FIG. 13, FIG. 14 and FIG. 15. The connection component includes a second limiting shaft 290, and the first limiting shaft 220 and the second limiting shaft 290 are connected with the connecting block 230, i.e., the first limiting shaft 220 and the second limiting shaft 290 may be inserted through the connecting block 230. The proximal end 333 of the locking tube abuts against a distal end 213 of the tube body 210, i.e., the locking component 330 and the tube body 210 may have the same diameter and the locking component 330 may be arranged at the distal end of the tube body 210. The tube body 210 includes a sliding groove 211, the second limiting shaft 290 is slidably arranged in the sliding groove 211. The sliding groove 211 includes a positioning portion 2143. When the connection component is in the first position, the first limiting shaft 220 is located in the connecting channel (i.e., the first groove portion 3321), and the second limiting shaft 290 is located in the positioning portion 2143.

In some embodiments, a second limiting structure 2142 is provided at a distal end of the positioning portion 2143, dividing the sliding groove 211 into the positioning portion 2143 and the sliding portion 2144. When the limiting shaft is in the positioning portion 2143, the relative position between the limiting shaft and the tube body 210 remains fixed. When the limiting shaft is in the sliding portion 2144, the limiting shaft may move along the sliding portion 2144 relative to the tube body 210.

In some embodiments, the sliding groove 211 further includes the sliding portion 2144 located at the proximal end of the positioning portion 2143, the positioning portion 2143 communicates with the sliding portion 2144, and a third limiting structure 2141 is arranged between the sliding portion 2144 and the positioning portion 2143.

In some embodiments, the sliding groove 211 further includes a second opening 2145 facing the distal end 2111 of the sliding groove 211.

Each of the second limiting structure 2142 and the third limiting structure 2141 is a structure protruding toward the inside of the sliding groove 211, and the positioning portion 2143 is formed between the second limiting structure 2142 and the third limiting structure 2141.

In some embodiments, the first limiting shaft 220 passes through the limiting groove 332 of the locking tube and the connecting block 230, while the second limiting shaft 290 passes through the sliding groove 211 of the tube body 210 and the connecting block 230. In some embodiments, the connecting block 230 is provided with a first shaft hole 233 and a second shaft hole 235; the first limiting shaft 220 passes through the limiting groove 332 and the first shaft hole 233, and the second limiting shaft 290 passes through the sliding groove 211 and the second shaft hole 235. By providing the connecting block 230 and inserting the first limiting shaft 220 and the second limiting shaft 290 through the connecting block 230, the connecting block 230 is coupled to both the locking component 330 and the tube body 210 through the first limiting shaft 220 and the second limiting shaft 290 respectively, thereby achieving connection between the tube body 210 and the locking component 330.

In some embodiments, a diameter of the first limiting shaft 220 and a diameter of the second limiting shaft 290 may be the same or different. Merely by way of example, the diameter of the first limiting shaft 220 may be less than or equal to the diameter of the second limiting shaft 290, which facilitates the subsequent separation process.

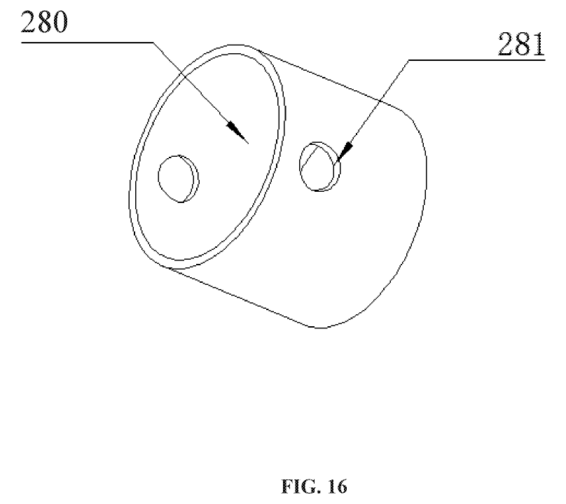

In some embodiments, as shown in FIG. 16, the connection component further includes an outer tube 280, the outer tube 280 is connected with the second limiting shaft 290. When the connection component is in the first position, the outer tube 280 simultaneously sleeves the locking tube and the tube body 210. When the connection component moves from the first position to the second position, an area where the outer tube 280 sleeves the locking tube decreases. When the connection component is in the second position, the outer tube 280 sleeves the tube body 210.

In some embodiments, the outer tube 280 is provided with two through holes 281, and the two through holes 281 are arranged opposite each other. The second limiting shaft 290 passes through the two sliding grooves 211 and the two through holes 281. The outer tube 280 may be positioned at the contact location between the tube body 210 and the locking component 330 and enclose the contact location. By enclosing the tube body 210 and the locking component 330 with the outer tube 280, deflection between the locking component 330 and the tube body 210 may be prevented.

In some embodiments, the second limiting shaft 290, under an external force, may be disengaged from the positioning portion 2143 and enter the sliding portion 2144 enclosed between the proximal end 2112 of the sliding groove 211 and the third limiting structure 2141 after passing the third limiting structure 2141. When the second limiting shaft 290 is located in the positioning portion 2143, a relative position between the second limiting shaft 290 and the tube body 210 is fixed. When the second limiting shaft 290 is located in the sliding portion 2144, the second limiting shaft 290 may move a certain distance along the first direction. The certain distance is a length of the sliding portion 2144 along the first direction. The second limiting shaft 290 passing the third limiting structure 2141 may refer to a structure of the third limiting structure 2141 being damaged by the second limiting shaft 290 under the external force, causing the third limiting structure 2141 to curl inward or outward, or causing the third limiting structure 2141 to be squeezed and deformed, becoming smooth and no longer having a limiting function.

In some embodiments, the first limiting shaft 220, under an external force, passes the first limiting structure 3322 to be disengaged from the first groove portion 3321 and may be disengaged from the guide groove 332 through the opening 3324, thereby releasing the connection between the locking component 330 and the tube body 210.

In some embodiments, after the first limiting shaft 220 separates from the guide groove 332, the first limiting shaft 220 may pass through the second opening 2145 under the external force, and pass the second limiting structure 2142, and enter the positioning portion 2143. Subsequently, under the external force, the first limiting shaft 220 may pass the third limiting structure 2141 to disengaged from the positioning portion 2143, and move into the sliding portion 2144.

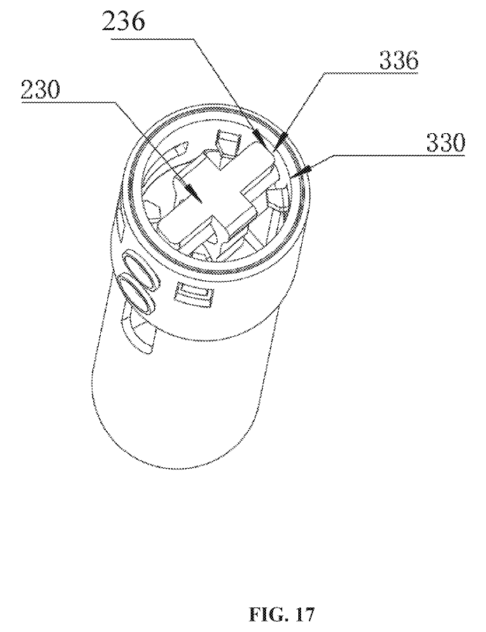

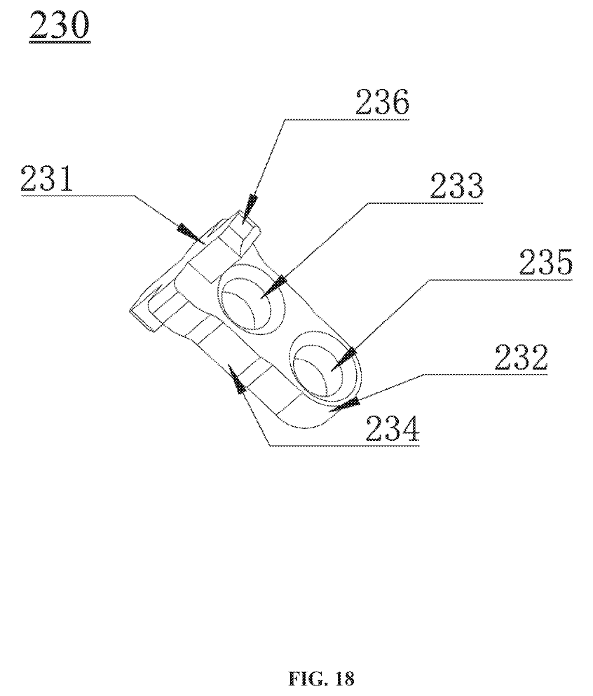

FIG. 17 is a schematic diagram illustrating an installation process of a connecting block according to some other embodiments of the present disclosure. FIG. 18 is a schematic diagram illustrating an exemplary connecting block according to some other embodiments of the present disclosure.

In some embodiments, as shown in FIG. 17 and FIG. 18, the connection component further includes a support structure 236. The support structure 236 is connected to the connecting block 230. The support structure 236 is configured to abut against an inner wall of the locking tube and/or an inner wall of the tube body 210 in the radial direction. In some embodiments, the support structure 236 is located at the distal end 231 of the connecting block 230 and protrudes from the connecting block 230 along the axial direction of the first shaft hole 233 and the second shaft hole 235.

By arranging the support structure 236 to fit against an inner wall 336 of the locking component 330, radial wobbling of the connecting block 230 relative to the tube body 210 may be prevented.

In some embodiments, as shown in FIG. 3 and FIG. 5, the locking component 330 is provided with at least one first force relief groove 334. For example, each of two sides of the guide groove 332 may be provided with one first force relief groove 334 to control a connection release force between the locking component 330 and the tube body 210. The connection release force between the locking component 330 and the tube body 210 refers to a minimum external force required to release a limit between the locking component 330 and the tube body 210, causing separation, relative motion between the locking component 330 and the tube body 210. The first force relief groove 334 refers to an elongated groove opened on one of the two sides of the guide groove 332. The first force relief groove 334 is configured to change material mechanical properties around the guide groove 332 to precisely regulate the connection release force. For example, the first force relief groove 334 may reduce the connection release force by decreasing a cross-sectional area of material on the two sides of the guide groove 332 to reduce local stiffness, thereby avoiding an excessively high release force due to excessive stiffness. The first force relief groove 334 may disperse local stress peaks generated during assembly or under force to prevent plastic deformation or fracture of the material due to excessive stress. In some embodiments, the first force relief groove 334 may guide material around the guide groove 332 to deform in a preset direction, ensuring a smooth and controllable separation process.

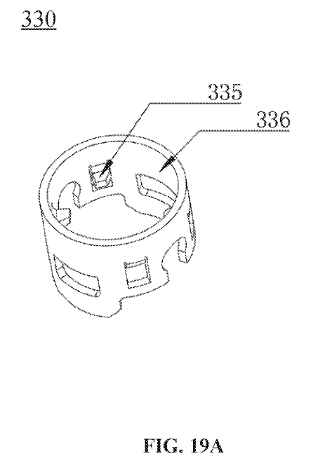

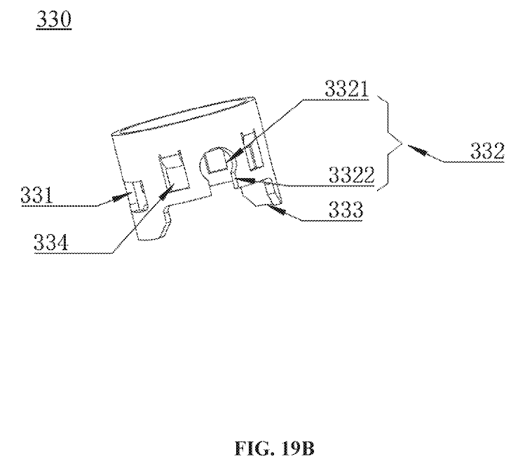

FIG. 19A is a schematic diagram illustrating an exemplary locking component according to some other embodiments of the present disclosure. FIG. 19B is a schematic diagram illustrating an exemplary locking component according to some other embodiments of the present disclosure.

In some embodiments, as shown in FIG. 19A and FIG. 19B, the locking component 330 further includes a stop member 335. The stop member 335 is formed by bending a portion of the side wall of the locking component 330 toward an inner side of the locking component 330, so that the stop member 335 projects from the inner wall 336 of the locking component 330 and simultaneously creates a notch structure in the locking component 330. The notch structure may perform the same function as the first force relief groove 334, and no further description is repeated here.

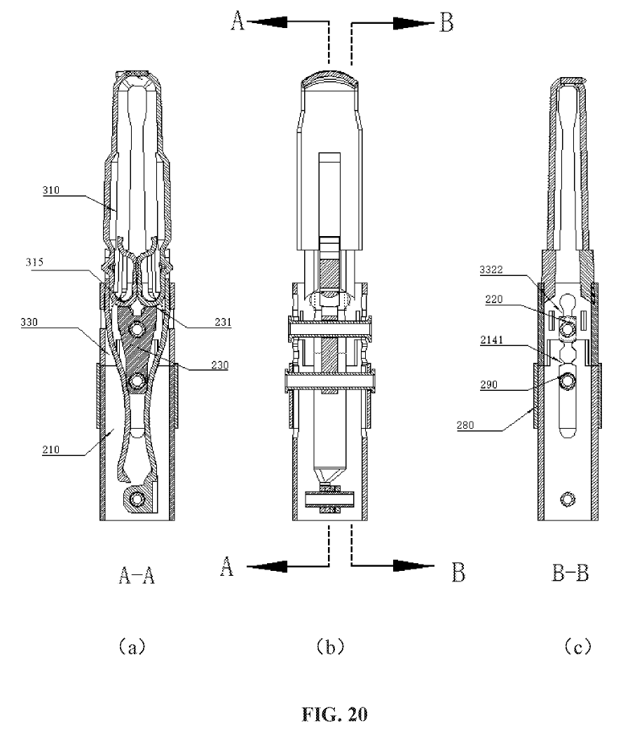

FIG. 20 is a schematic diagram illustrating a disassembly process of a locking component and an outer tube according to some other embodiments of the present disclosure, wherein (a) is a cross-sectional view along A-A in image (c) and (c) is a cross-sectional view along B-B in image (b).

Merely by way of example, as shown in FIG. 20, when the clip is in the closed state, the abutting portion 315 of the clip and a distal end 231 of the connection block 230 cooperate with each other. When the connection block 230 moves toward the proximal end of the clip instrument, thereby driving the first limiting shaft 220 to pass the first limiting structure 3322 and driving the second limiting shaft 290 to break through the third limiting protrusion 2141, thereby completing separation of the locking component 330 from the tube body 210. Meanwhile, the outer tube 280 is driven by the second limiting shaft 290 to move synchronously toward the proximal end of the outer tube 280, thereby gradually reducing an overlapping portion between the locking component 330 and the outer tube 280 until the locking component 330 and the outer tube 280 are completely separated, ensuring complete separation and avoids jamming that may affect operational feel.

In some embodiments of the present disclosure, the locking component 330 and the tube body 210 are connected by means of the limiting shaft and the connecting block 230, thereby achieving a reliable connection while ensuring that the tube body 210 and the locking tube may be separated smoothly and stably. Moreover, the locking component 330 and the tube body 210 do not interfere with each other in a direction of movement, making it possible to further reduce the length of the locking component 330.

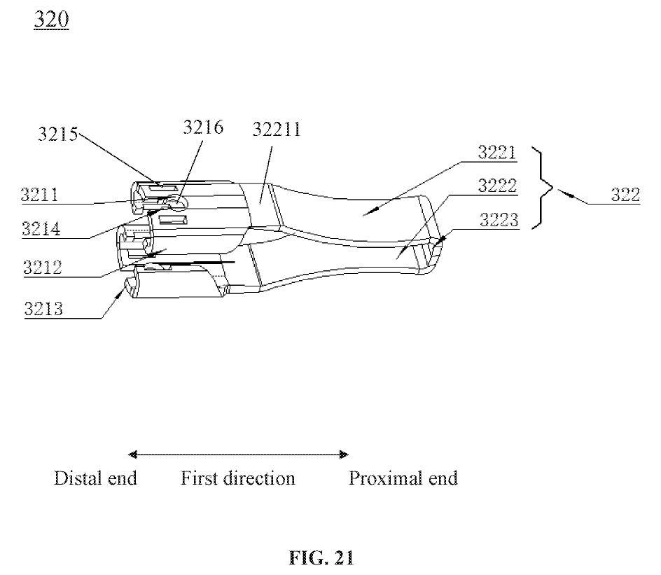

FIG. 21 is a schematic diagram illustrating an exemplary connection component according to some embodiments of the present disclosure.

In some embodiments, as shown in FIG. 21A, the clip further includes a connecting portion 320, the connecting portion 320 includes at least two connecting arms extending toward a distal end of the clip instrument, and each of the at least two connecting arms is releasably connected with one of the at least two clamping components 310.

The connecting portion 320 is configured to connect the at least two clamping components 310 and to drive the at least two clamping components 310 to move. In some embodiments, at least part of the connecting portion 320 is located inside the tube body 210, and the connecting portion 320 may move along the first direction so as to drive the at least two clamping components 310 to move toward the distal end of the clip instrument or the proximal end of the clip instrument along the first direction. In some embodiments, the connecting portion 320 is connected to the proximal end of the each of the at least two clamping components 310. When the at least two clamping components 310 are outside the tube body 210, the at least part of the connecting portion 320 extends out of the tube body 210 to connect with the at least two clamping components 310, and when the at least two clamping components 310 are inside the tube body 210, the connecting portion 320 is located inside the tube body 210.

In some embodiments, the connection between the connecting portion 320 and the at least two clamping components 310 may be released, so that the connecting portion 320 and the at least two clamping components 310 separate and the at least clamping components 310 may remain in a living body independently of the connecting portion 320. In some embodiments, after the connection portion 320 is separated from the at least two clamping components 310, the at least two clamping components 310 are separated from all other components of the clip instrument except the locking component 330. At this time, the locking component 330 maintains the at least two clamping components 310 in the closed state, so that the at least two clamping components 310 independently implement a clamping effect and remains in the living body to maintain wound closure.

In some embodiments of the present disclosure, the connecting portion 320 drives the two clamping components 310 to open and close, so that the at least two clamping components 310 themselves do not need to be provided with any opening/closing or movement-related structures, thereby shortening the length of the at least two clamping components 310 and thus reducing the length of the parts retained in the body and improving applicability.

In some embodiments, as shown in FIG. 3, FIG. 4A and FIG. 4B, the at least two clamping components 310 include a locked portion, the locking component 330 includes a locking portion, the locking portion is provided near a distal end of the locking component 330, and when the locking portion engages with the locked portion, the locking component 330 locks the at least two clamping components 310.

The locking portion is configured to connect with the two clamping components 310. In some embodiments, after the locking portion is connected with the two clamping components 310, the locking portion may lock the two clamping components 310 in the closed state.

The locking portion is provided near a distal end of the locking component 330 refers to that the locking portion is positioned on the locking component 330 at a location relatively closer to a distal end of the locking component 330, so that a connection position between the at least two clamping components 310 and the locking component 330 is nearer the distal end of the locking component 330. With this arrangement, while maintaining a stable connection between the locking portion and the at least two clamping components 310, the travel stroke of the at least two clamping components 310 is reduced, thereby shortening lengths of both the at least two clamping components 310 and the locking component 330.

In some embodiments, at least part of each of the at least two clamping components 310 is connected with the locking portion via a snap-fit connection, so that the locking portion locks the at least two clamping components 310.

In some embodiments, the locking component 330 is a tubular structure. The connecting portion 320 is movably arranged inside the locking component 330. The locking portion is a locking recess 331 opened inward on a tube wall of the tubular structure, and the locked portion is a locking protrusion 3111 facing the tube wall. The locking protrusion 3111 engages with the locking recess 331 via a snap-fit connection.

In some embodiments, a count of the locking recesses 331 matches a count of locking protrusions 3111. For example, if each of the two clamping components 310 has one locking protrusion 3111, the locking portion includes two locking recesses 331; if each of the two clamping components 310 has two locking protrusions 3111, the locking portion includes four locking recesses 331.

In some embodiments, the locking protrusion 3111 has an inclined ramp that faces a proximal end of the locking protrusion 3111, so that when the at least two clamping components 310 is moved toward the proximal end of the clip instrument, the locking protrusion 3111 may readily enter the locking recess 331.

In some embodiments of the present disclosure, by providing the locking recess 331, the locking component 330 engages with the at least two clamping components 310 via the snap-fit connection, offering a simple structure and stable engagement.

In some embodiments, each of the at least two clamping components 310 includes a clip blade 3100.

The clip blades 3100 are configured to cooperate with each other to achieve clamping. In some embodiments, the clip blade 3100 is a sheet-like structure. In some embodiments, a distal end of the clip blade 3100 is provided with a toothed structure capable of abutting and cooperating with a distal end of another clip blade 3100. The toothed structure helps stabilize mutual cooperation when the at least two clamping components 310 close and provides a force-bearing fulcrum for clamping. In some embodiments, the clip blade 3100 may be arc-shaped to match a shape inside the tube body 210. The arc shape facilitates movement within the tube body 210 and avoids forming sharp edges to prevent injury to the living body.

In some embodiments, an outer side of the clip blade 3100 is provided with a protection member 313. The clip instrument is used in cooperation with an endoscope and may be deployed within a device channel of the endoscope. The protection member 313 is configured to prevent an inner wall of the device channel of the endoscope from being scratched when the clip instrument bends.

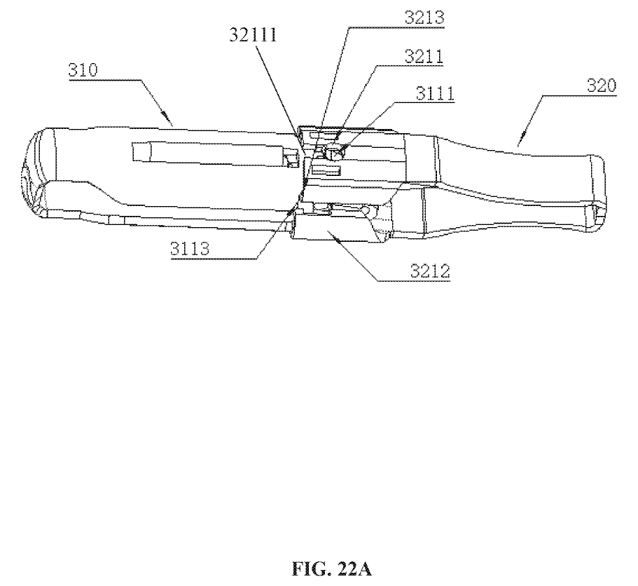

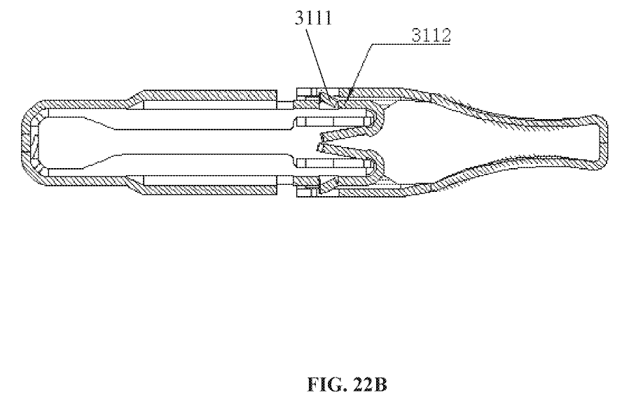

FIG. 22A is a schematic diagram illustrating connection between two clamping components and a connection component according to some embodiments of the present disclosure. FIG. 22B is a schematic diagram illustrating a cross-sectional view of a connection between two clamping components and a connection component according to some other embodiments of the present disclosure.

In some embodiments, as shown in FIG. 4A, FIG. 4B, FIG. 20, FIG. 22A, and FIG. 22B, each of the at least two clamping components 310 includes a first fixing portion. Each of the at least two connecting arms includes a second fixing portion. The each of the at least two clamping components 310 and the each of the at least two connecting arms are releasably connected through the first fixing portion and the second fixing portion.

In some embodiments, the first fixing portion is a protrusion provided on a side wall 3112 of the clamping component 310, and the second fixing portion is a recess 3216 provided on the connecting arm. In some embodiments, the first fixing portion and the second fixing portion may be releasably connected by any other practicable manners, such as the first fixing portion enveloping the second fixing portion, the second fixing portion enveloping the first fixing portion, riveting, pin connection, or hemming.

In some embodiments, the locking protrusion 3111 constitutes the first fixing portion, i.e., the locking protrusion 3111 is configured to connect with the locking component 330 and connect with the connecting portion 320.

In some embodiments, the clip blade 3100 of the at least two clamping components 310 may be a sheet-like structure. The distal end of the connecting arm may be an approximately elliptical hollow structure. A portion of the approximately elliptical hollow structure facing the inner side of the approximately elliptical hollow structure may be unenclosed. The elliptical hollow structure partially or completely envelops the proximal end of the clip blade 3100 of the at least two clamping components 310.

In some embodiments, the clamping component 310 includes an abutting portion 315, movement of the clip from the distal end of the clip instrument toward the proximal end the clip instrument causes the abutting portion 315 to drive the connection component to move from a first position to a second position. And after the connection component leaves the first position, the locking portion and the locked portion engage, in other words, after the locking component 330 separates from the tube body 210, the locking component 330 engages and connects with the clamping component 310. More descriptions regarding the abutting portion 315 may be found in the preceding description and related descriptions thereof.

In some embodiments, each of the at least two clamping components 310 includes an elastic portion 312. When the at least two clamping components 310 are in a closed state, the elastic portions 312 of the at least two clamping components 310 abut against each other.

In some embodiments, the clip blade 3100 is connected to the elastic portion 312 through the abutting portion 315, and the elastic portion 312 is located on an inner side of the clip blade 3100 at a position corresponding to where the locking protrusion 3111 is provided.

The elastic portion 312 is configured to provide a force along the radial direction of the tube body 210 toward the outside when the at least two clamping components 310 are closed. The force provided by the elastic portion 312 may be generated by deformation of two elastic portions 312 of the at least two clamping components 310 caused by mutual compression. For example, when the two clamping components 310 are closed, the elastic portions 312 of the two clamping components 310 contact each other, compress each other, and deform. Thus, the elastic portions 312 provide a force outward to the locking protrusion 3111 and the force drives the locking protrusion 3111 to move outward in the radial direction to engage the locking recess 331. Once the locking protrusion 3111 has engaged the locking recess 331, the force further promoting the stable connection between the locking protrusion 3111 and the locking recess 331.

In some embodiments, the elastic portion 312 may have a straight plate-like structure or an arc plate-like structure. Referring to FIG. 4A, in a natural state, a dimension of the elastic portion 312 along the radial direction of the tube body 210 is larger than a radius of the tube body 210. When the two clamping components 310 are closed, two elastic portions 312 of the two clamping components 310 abut against each other and undergo compressive deformation in a radial direction of the tube body 210, thereby generating a force directed outward in the radial direction of the tube body 210. In other embodiments, referring to FIG. 4B, the elastic portion 312 and the clip blade 3100 of the clamping component 310 may form an oval track-like shape. In a natural state, the dimension of the oval track-like shape along the radial direction of the tube body 210 is slightly larger than the radius of the tube body 210. When the two clamping components 310 are closed, two oval track-like shapes corresponding to the two clamping components 310 abut against each other and may generate a relatively large force directed outward in the radial direction, ensuring reliable locking of the clamping component 310 by the locking protrusion 3111. The elastic portion 312 may also have any other feasible structure, as long as the elastic portion 312 provides a stable outward-acting force meeting requirements for the locking protrusion 3111.

In some embodiments of the present disclosure, by providing the locking recess 331 and the locking protrusion 3111 to cooperate, the locking component 330 engages with the at least two clamping components 310 via the snap-fit connection. By providing the elastic portion 312, connection stability may be enhanced.

In some embodiments, as shown in FIG. 21, the connecting portion 320 includes a first limiting portion 322. The first limiting portion 322 includes at least a first side portion 3221, a second side portion 3222, and a first proximal portion 3223. The first proximal portion 3223 connects the proximal end of the first side portion 3221 and the proximal end of the second side portion 3222. The first side portion 3221 and the second side portion 3222 form the connecting arms. The first limiting portion 322 may also include more than two side portions, a count of the side portions matching a count of the clamping components 310 so as to connect with two or more clamping components 310.

In some embodiments, the first limiting portion 322 further includes end side portions 32211 located at the distal ends of the side portions. For example, two end side portions 32211 are located at a distal end of the first side portion 3221 and a distal end of the second side portion 3222. In the natural state, the end side portions 32211 are positioned farther outward than the side portions (e.g., the first side portion 3221 and the second side portion 3222). The connecting portion 320 may abut against the inner wall 336 of the locking component 330 via the end side portions 32211, thereby causing at least two clamping components 310 to switch from the open state to the closed state. In some embodiments, when the at least two clamping components 310 are in the open state, the connecting arms move from the distal end of the clip instrument toward the proximal end of the clip instrument, converting the at least two clamping components 310 from the open state to the closed state; when the end side portions 32211 abut against the locking component 330, the at least two clamping components 310 are in the closed state.

In some embodiments, the first limiting portion 322 is connected to a transmission member of the clip instrument. The transmission member is configured to transmit power. For example, the transmission member is a rigid column. The rigid column may be connected to a manual driving member and/or an electric driving member (e.g., a motor). Therefore, the rigid column may transmit a moving force or a rotating force of the driving member to drive the first limiting portion 322 to perform a corresponding motion, thereby driving the connecting portion 320 to perform a corresponding motion.

The first side portion 3221 and the second side portion 3222 may be plate-like structures with a certain curvature, so that distal ends of the first side portion 3221 and the second side portion 3222 are positioned farther outward than proximal ends of the first side portion 3221 and the second side portion 3222, allowing the distal ends of the first side portion 3221 and the second side portion 3222 to connect with the end side portions 32211. The first limiting shaft 220 may be located between the first side portion 3221 and the second side portion 3222. When the connecting portion 320 moves a certain distance toward the distal end of the clip instrument, the first proximal portion 3223 is stopped by the first limiting shaft 220, thereby limiting a range of distal movement of the connecting portion 320.

In some embodiments, when the two clamping components 310 are in the closed state, a proximal end of the abutting portion 315 abuts against a distal end of the first limiting shaft 220, to limit the connecting portion 320 from continuing to move toward the proximal end of the clip instrument. When the two clamping components 310 are in a maximum open state, the first proximal end portion 3223 may be a sheet-like structure. A surface of the first proximal part 3223 facing a distal end of the first proximal end portion 3223 abuts against a proximal end of the first limiting shaft 220, to limit the connecting portion 320 from continuing to move toward the distal end, thereby preventing the connecting portion 320 from being disengaged from the tube body 210.

In some embodiments, as shown in FIG. 6 and FIG. 7, the connecting block 230 may be located between the first side portion 3221 and the second side portion 3222, with side walls 234 of the connecting block 230 respectively opposing the first side portion 3221 and the second side portion 3222. That is, an inward facing surface of the first side portion 3221 faces one side wall 234 of the connecting block 230, and an inward facing surface of the second side portion 3222 faces the other side wall 234 of the connecting block 230, so that the connecting block 230 may limit a range of movement of the connecting portion 320.

In some embodiments, when the two clamping components 310 are in the closed state, a proximal end of the abutting portion 315 abuts against a distal end of the connecting block 230, to limit the travel of the connecting portion 320 during the separation of the tube body 210 from the locking component 330. So that the separation of the tube body 210 from the locking component 330 is completed just before or simultaneously with the locking of the locking component 330 to the two clamping components 310. In some embodiments, when the two clamping components 310 are in a maximum open state. A distal end 3225 of the first proximal end portion 3223 abuts against a proximal end 232 of the connecting block 230, to limit the connecting portion 320 from continuing to move toward the distal end of the clip instrument, thereby preventing the connecting portion 320 from being disengaged from the tube body 210.

In some embodiments, as shown in FIG. 21, the second fixing portion is a through-opening. When the connecting portion 320 drives the at least two clamping components 310 toward the proximal end of the clip instrument and the locking protrusion 3111 moves along the first direction into alignment with the locking recess 331, the locking protrusion 3111 passes through the through-opening and enters the locking recess 331. The connecting portion 320 then continues to move toward the proximal end of the clip instrument, the cooperating portion 3214 deforms or fails, and the connection between the first fixing portion and the second fixing portion is thereby released.

In some embodiments, when the connecting portion 320 drives the at least two clamping component 310 toward the proximal end of the clip instrument and the locking protrusion 3111 moves along the first direction to the locking recess 331, distal displacement of the at least two clamping components 310 is restricted. The connecting portion 320 continues to move toward the proximal end of the clip instrument, the connection between the first fixing portion and the second fixing portion is released, and the locking protrusion 3111 enters the locking recess 331.

Reference to FIG. 21, FIG. 22A and FIG. 22B, in some embodiments, the through-opening may include a fixing groove 3211 extending along the first direction. The fixing groove 3211 includes a third opening 32111 facing a distal end of the fixing groove 3211, and a cooperating portion 3214 is provided inside the fixing groove 3211. The locking protrusion 3111 may engage with the fixing groove 3211 via the snap-fit connection.

In some embodiments, the connecting portion 320 includes an arcuate bent edge 3212 that may envelop the clip blade 3100, thereby enhancing connection stability between the at least two clamping components 310 and the connecting portion 320 and ensuring overall smoothness of the clip instrument.

In some embodiments, the clamping component 310 includes a limiting surface 3113 facing the proximal end. The limiting surface 3113 may abut against and cooperate with the distal end 3213 of the connecting portion 320 to limit proximal movement of the clamping component 310 relative to the connecting portion 320.

The cooperating portion 3214 refers to a structure protruding toward an interior of the fixing groove 3211. In some embodiments, the cooperating portion 3214 and a proximal end of the fixing groove 3211 enclose the recess 3216. When the locking protrusion 3111 is located in the recess 3216, the proximal end of the fixing groove 3211 and the cooperating portion 3214 limit the first protrusion 3111, fixing a relative position between the first protrusion 3111 and the connecting portion 320.

In some embodiments, the cooperating portion 3214 may be broken or deformed by the locking protrusion 3111 under an external force, enabling the locking protrusion 3111 to be disengaged from the recess 3216 and to be disengaged from the fixing groove 3211 through the third opening 32111, thereby releasing the connection between the two clamping component 310 and the connecting portion 320.

In some embodiments, the fixing groove 3211 further includes a slot segment. The slot segment is located between the third opening 32111 and the cooperating portion 3214. After the locking protrusion 3111 may be disengaged from the recess 3216, the locking protrusion 3111 may first enter the slot segment and then pass through the third opening 32111. Through a buffering and guiding effect of the slot segment, stability and applicability of the clip instrument may be improved.

In some embodiments, at least one second force relief groove 3215 is provided on the each of the connecting portion 320. For example, one second force relief groove 3215 may be provided on each of two sides of the fixing groove 3211. A function of the second force relief groove 3215 is similar to a function of the first force relief groove 334, and is not repeated here.

Merely by way of example, an opening and closing process of the clip instrument, a separation process between the at least two clamping components 310 and the connecting portion 320, and a locking process with the locking component 330 are described below with reference to the accompanying drawings.

The connecting portion 320 moves toward the distal end of the clip instrument, driving the at least two clamping components 310 to move toward the distal end of the clip instrument. The first side portion 3221 and the second side portion 3222 of the connecting portion 320 open outward under an action of the side wall 234 of the connection block 230, thereby driving the at least two clamping components 310 to open.

When the two clamping components 310 opens to the maximum open state, the first proximal end portion 3223 of the connecting portion 320 contacts a proximal end 232 of the connection block 230, preventing the connecting portion 320 from continuing to move toward the distal end of the clip instrument as shown in FIG. 7.

The connecting portion 320 moves toward the proximal end of the clip instrument, driving the at least two clamping components 310 to move toward the proximal end of the clip instrument. With cooperation of the locking component 330, the two clamping components 310 gradually closes. When the two clamping components 310 are completely closed, i.e., in the closed state, the abutting portion 315 abuts against the distal end 231 of the connecting block 230, while the elastic portions 312 abut against each other and apply a force outward as shown in FIG. 9.

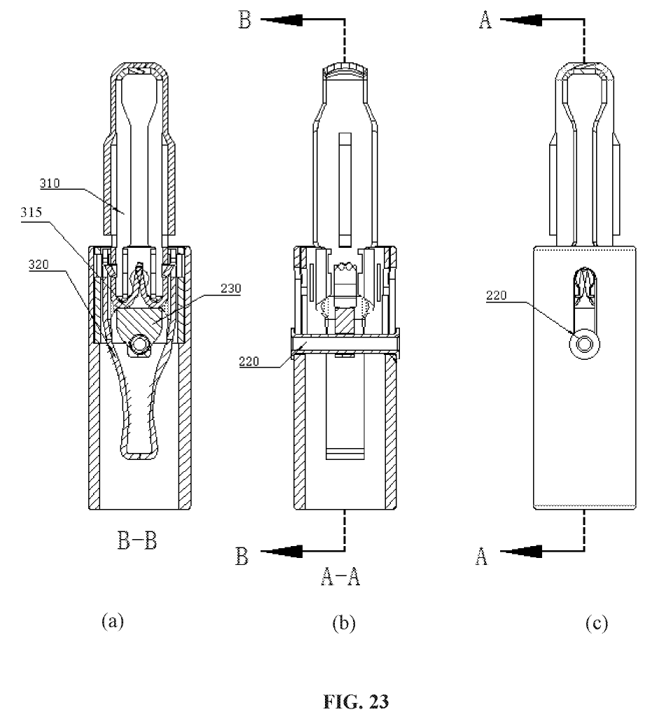

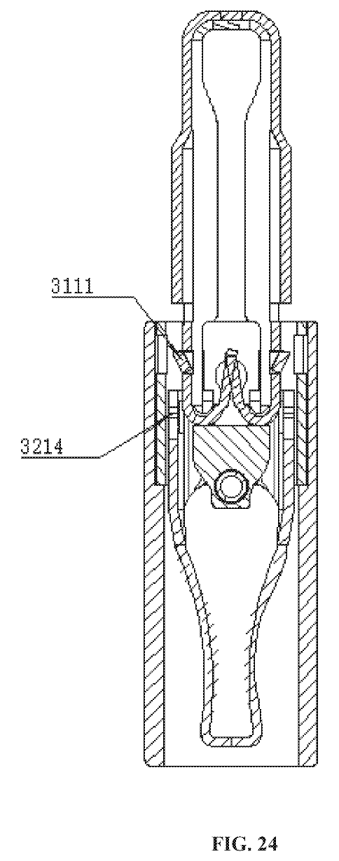

FIG. 23 is a schematic diagram illustrating a disassembly process of two clamping components and a connection component according to some embodiments of the present disclosure, wherein (b) is a cross-sectional view taken along A-A in (c), and (a) is a cross-sectional view taken along B-B in (b). FIG. 24 is a schematic diagram illustrating a disassembly process of two clamping components and a connection component according to some embodiments of the present disclosure.

After the locking component 330 is separated from the tube body 210, the connection portion 320 continues to move toward the proximal end of the clip instrument. Under an action of the abutting portion 315, the connecting block 230 and the first limiting shaft 220 also move toward the proximal end of the clip instrument until the first limiting shaft 220 contacts the proximal end 2112 of the sliding groove 211, and the at least two clamping components 310 stop moving due to force as shown in FIG. 23. The connection portion 320 continues to move toward the proximal end of the clip instrument, causing the locking protrusion 3111 to pass over the cooperating portion 3214, achieving separation between the connection portion 320 and the at least two clamping components 310. See FIG. 23.

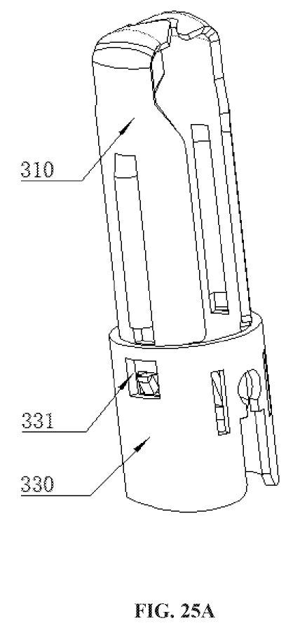

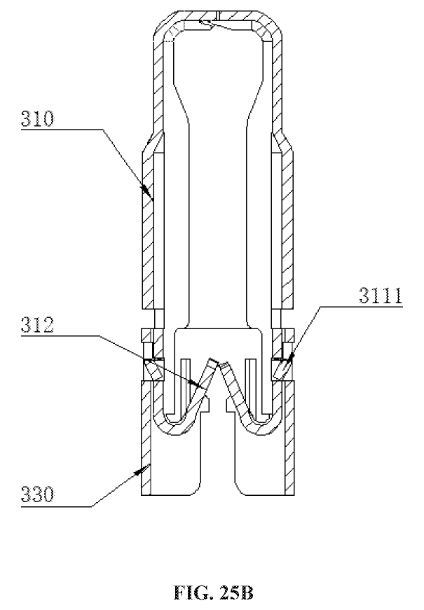

FIG. 25A is a schematic diagram illustrating a locking process of two clamping components and a locking component according to some embodiments of the present disclosure. FIG. 25B is a schematic diagram illustrating a sectional view of FIG. 25A according to some embodiments of the present disclosure.

Referring to FIG. 25A and FIG. 25B, after the at least two clamping components 310 and the connecting portion 320 are completely separated, under an elastic force of the elastic portion 312, causing the locking protrusion 3111 to enter the locking recess 331, achieving locking between the at least two clamping components 310 and the locking component 330.

In some embodiments of the present disclosure, by setting a connection structure between the each of the at least two clamping components 310 and the connection component and a connection structure as the same structure (i.e., the locking protrusion 3111), a structure of the clip instrument is simplified, reliability is improved, cost is reduced, and a length of the at least two clamping components 310 is further reduced. Separation is achieved by the locking protrusion 3111 passing over the cooperating portion 3214, ensuring that after the at least two clamping components 310 and the connection component are separated, there are no other force application points, thereby ensuring smooth separation between the at least two clamping components 310 and the connection component. By providing the elastic portion 312, the locking protrusion 3111 may enter the locking recess 311 of the locking component 330 under an action of the elastic portion 312, achieving locking between the at least two clamping components 310 and the locking component 330 while the connection component is separated from the tube body 210. Operation is simplified, force feedback is unified, and operation feel is improved.

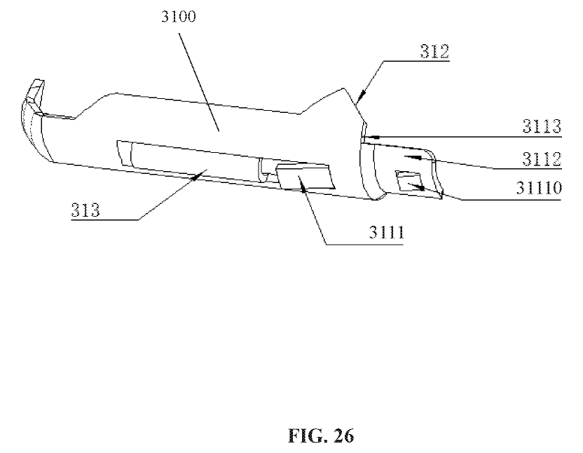

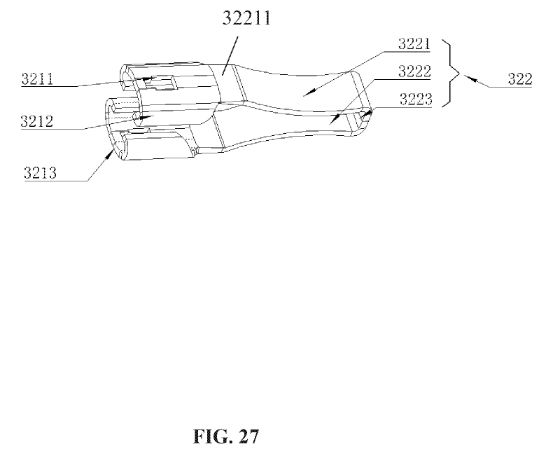

FIG. 26 is a schematic diagram illustrating an exemplary clamping component according to some other embodiments of the present disclosure. FIG. 27 is a schematic diagram illustrating an exemplary connection component according to some other embodiments of the present disclosure.

In some embodiments, as shown in FIG. 26 and FIG. 27, each of the at least two clamping components 310 further includes a connecting protrusion 31110, the locking protrusion 3111 is provided at a more distal end of the each of the at least two clamping components 310 compared to the connecting protrusion 31110, and the connecting protrusion 31110 constitutes the first fixing portion. The locking protrusion 3111 may engage with the locking recess 331 of the locking component 330 via the snap-fit connection. The connecting protrusion 31110 may engage with the fixing slot 3211 of the connecting component 320 via the snap-fit connection. It should be noted that the connecting component 320 of the present embodiment is structurally similar to that of the aforementioned embodiments. The fixing slot 3215 in the present embodiment is a closed slot without an open end.

In some embodiments of the present disclosure, configuring the fixing slot 3211 as a complete slot rather than an open slot may prevent premature separation of the at least two clamping components 310 and the connecting portion 320 due to excessive force during tissue clamping process of the at least two clamping components 310. The at least two clamping components 310 are provided with the locking protrusion 3111 and the connecting protrusion 31110 respectively, which may respectively achieve locking between the at least two clamping components 310 and the locking component 330 and separation between the at least two clamping components 310 and the connecting portion 320, thereby enhancing product reliability.

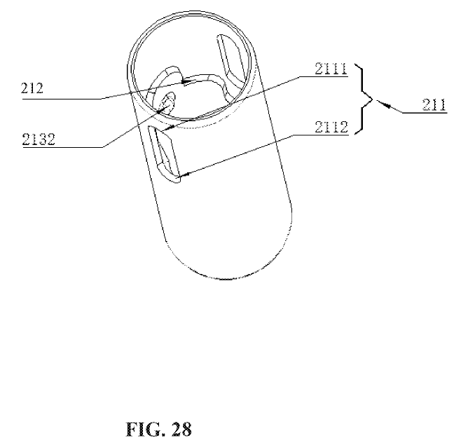

FIG. 28 is a schematic diagram illustrating an exemplary tube body according to some other embodiments of the present disclosure.

In some embodiments, as shown in FIG. 28, an inner wall of the tube body 210 is provided with the limiting protrusion 2132 matching the connecting protrusion 31110, and the limiting protrusion 2132 applies a force toward an interior of the tube body 210 on the connecting protrusion 31110.

Merely by way of example, when the connecting protrusion 31110 is engaged in the fixing slot 3211, the connecting portion 320 moves until the limiting protrusion 2132 is located outside the connecting protrusion 31110. The limiting protrusion 2132 applies a force on the connecting protrusion 31110 along the radial direction of the tube body 210 toward the interior of the tube body 210, squeezing the connecting protrusion 31110 and being disengaged from the fixing slot 3211 to achieve separation between the at least two clamping components 310 and the connecting portion 320.

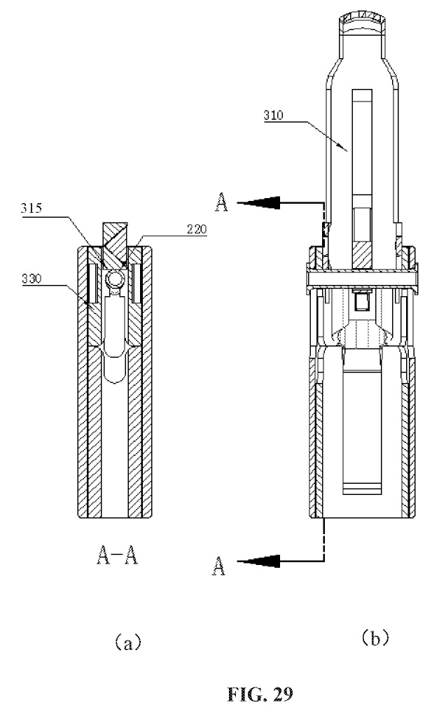

FIG. 29 is a schematic diagram illustrating a closed state of a clamping component according to some other embodiments of the present disclosure, wherein (a) is a cross-sectional view taken along A-A in (b).

Merely by way of example, referring to FIG. 29, similar to the implementation shown in the aforementioned FIG. 8, When the at least two clamping components 310 are in the closed state, the proximal end of the abutting portion 315 abuts against the distal end 231 of the connecting block 230.

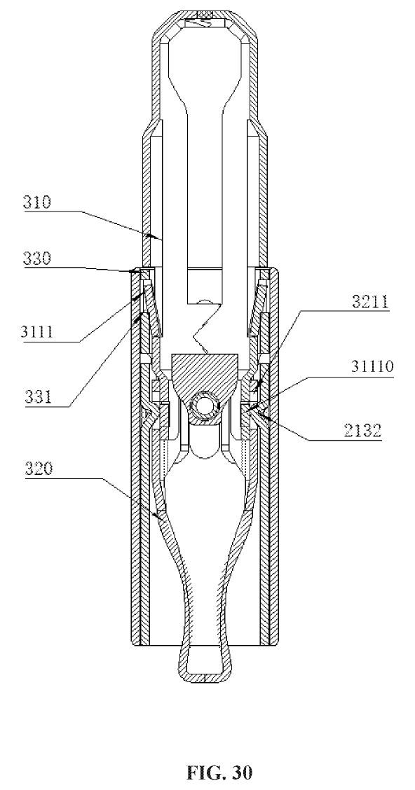

FIG. 30 is a schematic diagram illustrating a locking process of connection component and a locking component according to some other embodiments of the present disclosure.

Merely by way of example, the following describes the separation process between the at least two clamping components 310 and the connecting portion 320 and the locking process between the at least two clamping components 310 and the locking component 330 of the clip instrument in conjunction with FIG. 30.

After the locking component 330 separates from the tube body 210, the connecting portion 320 continues to move toward the proximal end of the clip instrument, driving the locking protrusion 3111 into the locking recess 331 to achieve locking between the connecting portion 320 and the locking component 330. Simultaneously, the connecting protrusion 31110 is deformed by being squeezed by the limiting protrusion 2132, causing the connecting protrusion 31110 to release from the fixing slot 3211 and achieving separation between the at least two clamping components 310 and the connecting portion 320 as shown in FIG. 30.

In some embodiments of the present disclosure, by separately arranging the connection structures on the at least two clamping components 310 for connecting to the connection portion 320 and the locking component 330, the at least two clamping components 310 may first lock with the locking component 330 and then separate from the connection component 320. This avoids a situation where the at least two clamping components 310 cannot lock with the locking component 330 after separating from the connection portion 320, ensuring product reliability.

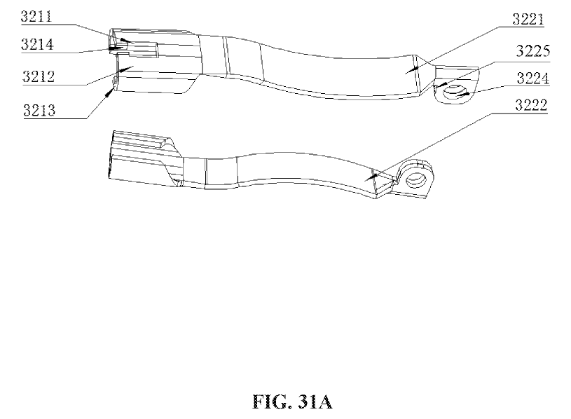

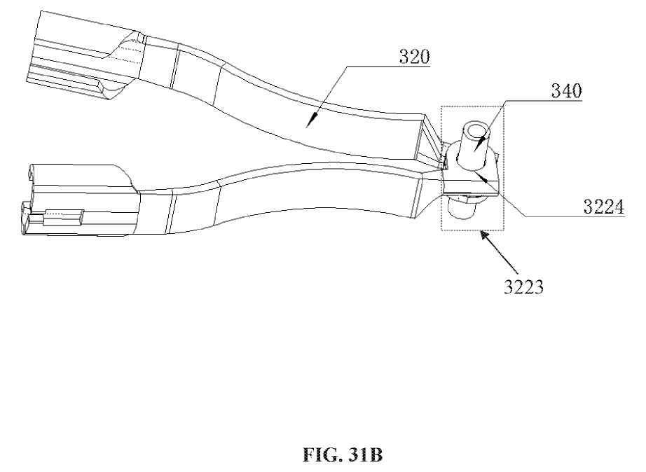

FIG. 31A is a schematic diagram illustrating an exemplary connection component according to some other embodiments of the present disclosure. FIG. 31B is a schematic diagram illustrating connection of a connection component according to some other embodiments of the present disclosure.

In some embodiments, as shown in FIG. 31A and FIG. 31B, the first limiting portion 322 includes a first side portion 3221, a second side portion 3222, and a first proximal end portion 3223. A distal end of the first side portion 3221 and a distal end of the second side portion 3222 are respectively connected to the two second fixing portions. A proximal end of the first side portion 3221 and a proximal end of the second side portion 3222 are rotatably connected by a pin shaft 340. Merely by way of example, the proximal end of the first side portion 3221 and the proximal end of the second side portion 3222 are each provided with a mounting hole 3224, and the pin shaft 340 is inserted through the mounting hole 3224 so that the first side portion 3221 and the second side portion 3222 may rotate about the pin shaft 340 to drive the opening and closing of the at least two clamping components 310. The first proximal portion 3223 is constituted by the mounting hole 3224 and the pin shaft 340.

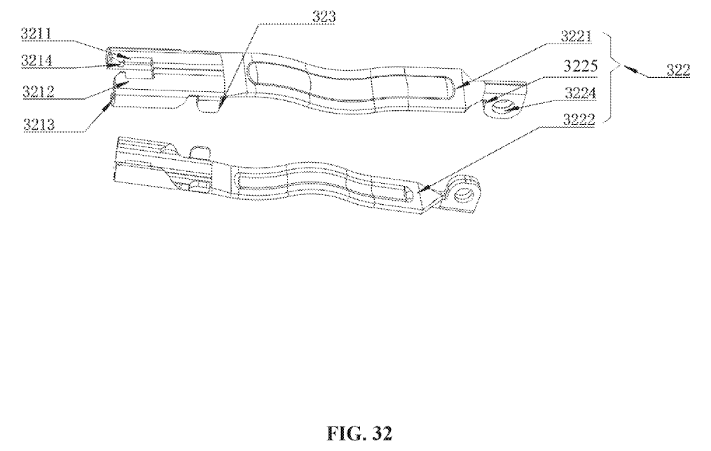

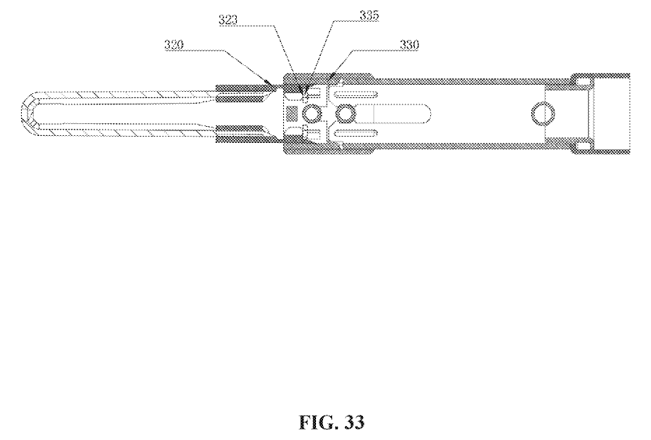

FIG. 32 is a schematic diagram illustrating an exemplary connection component according to some other embodiments of the present disclosure. FIG. 33 is a schematic diagram illustrating connection of a connection component according to some other embodiments of the present disclosure.

In some embodiments, as shown in FIG. 32, each of the at least two clamping components 310 further includes a bent portion 323. The bent portion 323 is disposed adjacent the distal end of the connecting portion 320 and protrudes toward an inner side of the at least two clamping components 310.

In some embodiments, as shown in FIG. 33, when the two clamping components 310 are in the closed state, the bent portion 323 may cooperate with the stop member 335 of the locking component 330, that is, a proximal end of the bent portion 323 may abut a distal end of the stop member 335 to provide a limit stop. This helps narrow the force-distribution window required for overall function of the clip instrument and reduces the likelihood of force-profile overlap among the steps of separating the locking component 330 from the tube body 210, locking the at least two clamping components 310 to the locking component 330, and separating the connecting portion 320 from the at least two clamping components 310, thereby improving product reliability.

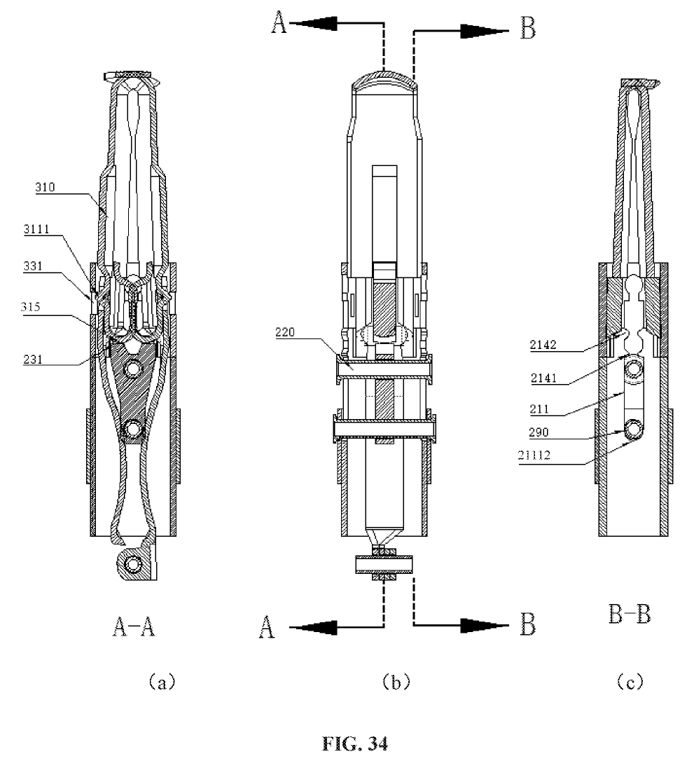

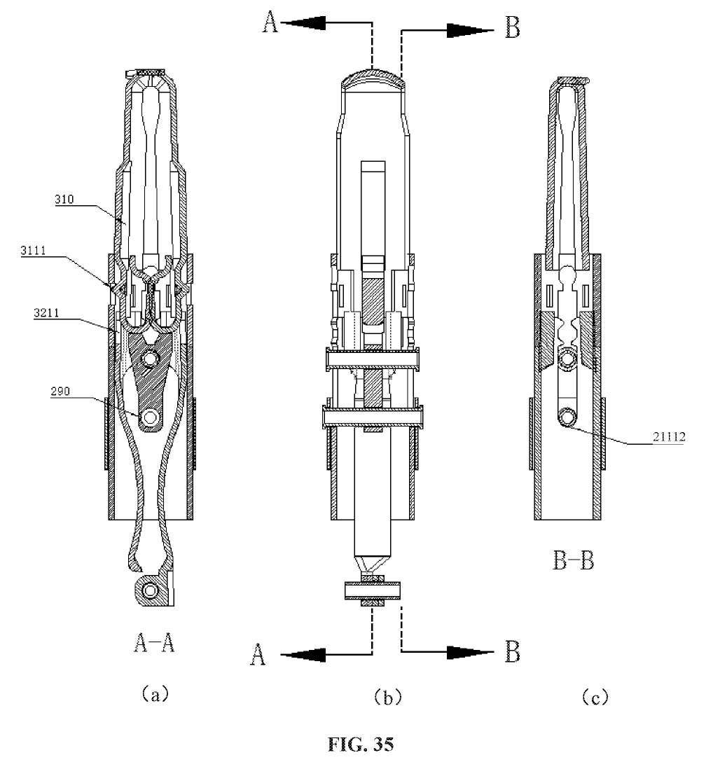

FIG. 34 is a schematic diagram illustrating a locking process of two clamping components according to some other embodiments of the present disclosure, wherein (a) is a cross-sectional view taken along A-A in (b), and (c) is a cross-sectional view taken along B-B in (b). FIG. 35 is a schematic diagram illustrating a disassembly process of two clamping components and a connection component according to some other embodiments of the present disclosure, wherein (a) is a cross-sectional view taken along A-A in (b), and (c) is a cross-sectional view taken along B-B in (b).

Merely by way of example, the following description, with reference to FIG. 34 and FIG. 35, explains a separate process and a lock process of the at least two clamping components 310 of the clip instrument from the connecting portion 320 and locking the at least two clamping components 310 with the locking component 330.

With reference to FIG. 34, after the locking component 330 and the outer tube 280 are separated, the connecting portion 320 continues to move toward the proximal end of the clip instrument, causing the abutting portion 315 to push the distal end 231 of the connecting block 230 to move toward the proximal end of the clip instrument. At this time, the first limiting shaft 220 breaks through the second limiting structure 2142 and the third limiting structure 2141, moving from a distal end 21111 of the sliding portion 2144 of the sliding groove 211 into the sliding portion 2144, until the second limiting shaft 290 contacts a proximal end 21112 of the sliding portion 2144 of the sliding groove 211, reaching a locking position. At this time, the locking protrusion 3111 and the locking recess 331 cooperate to complete locking of the at least two clamping components 310.

With reference to FIG. 35, after the at least two clamping components 310 is locked, the connecting portion 320 continues to move toward the proximal end of the clip instrument0. Because the second limiting shaft 290 contacts the proximal end 21112 of the sliding portion 2144 of the sliding groove 211, the at least two clamping components 310 cannot continue to move toward the proximal end of the clip instrument thereby causing the locking protrusion 3111 and the fixing slot 3211 to separate.

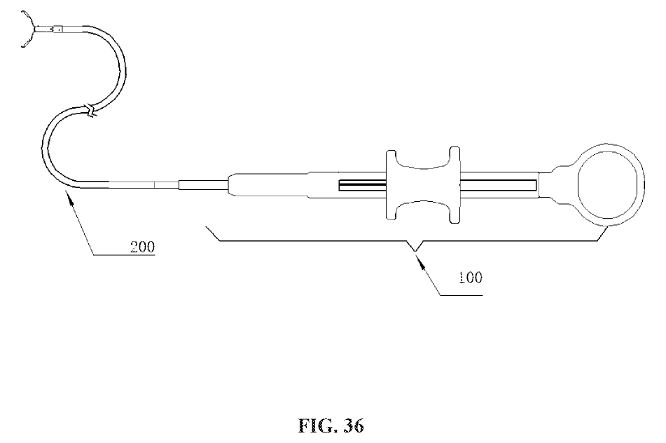

FIG. 36 is a schematic diagram illustrating a clip instrument according to some embodiments of the present disclosure.

In some embodiments, as shown in FIG. 36, the clip instrument further includes a handle portion 100 and a sheath tube portion 200. The handle portion 100 is a control component for medical personnel to operate the clip instrument. By using the handle portion 100, the medical personnel may drive movement of an internal structure of the clip instrument through external force, thereby performing a corresponding medical operation.

The sheath tube portion 200 serves as a channel of the clip instrument for entry and exit of other medical instruments. In some embodiments, the sheath tube portion 200 is connected to the tube body 210. Merely by way of example, as shown in FIG. 2, the sheath tube portion 200 includes a main body and a spring tip 240. The spring tip 240 is connected to a distal end of the main body. The proximal end of the tube body 210 engages with a flange of the spring tip 240 by a flanging 282 via the snap-fit connection, thereby achieving a rotatable connection of the sheath tube portion 200 relative to the tube body 210.

In some embodiments, the clip instrument further includes a transmission member, the transmission member is connected with the handle portion 100 and extends through the tube body 210. In some embodiments, the transmission member is connected with a proximal end of the connecting portion 320 to drive the connecting portion 320 to rotate and/or move along the first direction.

Merely by way of example, as shown in FIG. 2, the transmission member includes a connecting post 260 and a torque member 270. A distal end of the connecting post 260 may pass through a slot at the proximal end of the connecting portion 320. A proximal end of the connecting post 260 is fixedly connected to the torque member 270. The torque member 270 is connected to the handle portion 100. By pushing or pulling the torque member 270 via the handle portion 100, the connecting portion 320 may be driven to move along the first direction. By rotating the torque member 270 via the handle portion 100, the connecting portion 320 may be driven to rotate, and further the tube body 210 may be driven to rotate.

In some embodiments, the torque member 270 is a filamentous structure. An exterior of the torque member 270 may be covered with a protective tube 250 to protect the torque member 270 and ensure an effect of power transmission.

In some embodiments of the present disclosure may conveniently achieve control of the clip instrument by providing the handle portion and the sheath tube portion.

The foregoing description has set forth basic concepts. It is apparent to those skilled in the art that the foregoing detailed disclosure is merely exemplary and does not constitute a limitation on the present disclosure. Although not explicitly stated herein, those skilled in the art may make various modifications, improvements, and amendments to the present disclosure. Such modifications, improvements, and amendments are suggested in the present disclosure. Therefore, such modifications, improvements, and amendments still fall within the spirit and scope of the exemplary embodiments of the present disclosure.

In some embodiments, numbers describing quantities of components or properties are used. It should be understood that such numbers configured to describe the embodiments are, in some examples, modified by the modifiers "approximately," "about," or "substantially." Unless otherwise stated, "approximately," "about," or "substantially" indicates that the stated number allows a variation of ±20%. Accordingly, in some embodiments, numerical parameters used in the specification and claims are approximate values. The approximate values may vary according to characteristics required by individual embodiments. In some embodiments, numerical parameters should consider specified significant digits and adopt a general manner of digit retention. Although numerical ranges and parameters used to confirm the breadth of scope in some embodiments of the present disclosure are approximate values, in specific embodiments, setting of such numerical values is as precise as possible within a feasible range.

For each patent, patent application, patent application publication, and other material, such as articles, books, specifications, publications, documents, etc., cited in the present disclosure, the entire contents thereof are hereby incorporated by reference into the present disclosure. Excluded are application history documents inconsistent with or conflicting with the content of the present disclosure, and also excluded are documents (currently or later appended to the present disclosure) that limit the broadest scope of the claims of the present disclosure. It should be noted that if descriptions, definitions, and/or usage of terms in the attached materials of the present disclosure are inconsistent with or conflict with those in the present disclosure, the descriptions, definitions, and/or usage of terms in the present disclosure prevail.

Finally, it should be understood that the embodiments described in the present disclosure are only used to illustrate principles of the embodiments of the present disclosure. Other variations may also fall within the scope of the present disclosure. Therefore, by way of example and not limitation, alternative configurations of the embodiments of the present disclosure may be considered consistent with the teachings of the present disclosure. Accordingly, the embodiments of the present disclosure are not limited to the embodiments explicitly introduced and described in the present disclosure.

What is claimed is:

1. A clip instrument, comprising:
a tube body;
a clip, comprising at least two clamping components;
a locking component, releasably connected with the tube body and capable of locking the at least two clamping components;
a connection component, slidably connected with the tube body along a first direction and capable of moving relative to the locking component along the first direction.

2. The clip instrument according to claim 1, wherein:
the locking component includes a connecting channel, and the connection component includes a first limiting shaft;
when the connection component is in a first position, the first limiting shaft is located within the connecting channel; when the connection component is in a second position, the first limiting shaft disengages from the connecting channel.

3. The clip instrument according to claim 2, wherein:
the locking component is provided with a guiding groove, the guiding groove includes a first groove portion and a second groove portion, the second groove portion is located at a proximal end of the first groove portion, and a first limiting structure is provided between the second groove portion and the first groove portion, wherein the first groove portion constitutes the connecting channel.

4. The clip instrument according to claim 1, wherein:
the clip includes an abutting portion, and movement of the abutting portion toward a proximal end of the clip instrument drives the connection component to move toward the proximal end of the clip instrument.

5. The clip instrument according to claim 3, wherein:
the locking component is a locking tube, the guiding groove is provided at a proximal end of the locking tube, and the guiding groove has an opening facing the proximal end of the locking tube.

6. The clip instrument according to claim 5, wherein:
at least a portion of the locking tube is located inside the tube body, the tube body includes a sliding groove, and the guiding groove at least partially overlaps with the sliding groove.

7. The clip instrument according to claim 5, wherein:
the connection component includes a connecting block and a second limiting shaft, the first limiting shaft and the second limiting shaft are connected with the connecting block, the proximal end of the locking tube abuts against a distal end of the tube body, the tube body includes a sliding groove, the second limiting shaft is slidably arranged in the sliding groove, the sliding groove includes a positioning portion, when the connection component is in the first position, the first limiting shaft is located in the connecting channel, and the second limiting shaft is located in the positioning portion.

8. The clip instrument according to claim 7, wherein:
the connection component further includes an outer tube, the outer tube is connected with the second limiting shaft, when the connection component is in the first position, the outer tube simultaneously sleeves the locking tube and the tube body, and when the connection component moves from the first position to the second position, an area where the outer tube sleeves the locking tube decreases.

9. The clip instrument according to claim 1, wherein:
the clip further includes a connecting portion, the connecting portion includes at least two connecting arms extending toward a distal end of the clip instrument, and each of the at least two connecting arms is releasably connected with one of the at least two clamping components.

10. A clip instrument, characterized by comprising:
a tube body;
a clip, comprising at least two clamping components and a connecting portion, the connecting portion includes at least two connecting arms extending toward a distal end of the clip instrument, and each of the at least two connecting arms is releasably connected with one of the at least two clamping components;
a locking component, releasably connected with the tube body and capable of locking the at least two clamping components.

11. The clip instrument according to claim 10, wherein:
the at least two clamping components include a locked portion, the locking component includes a locking portion, the locking portion is provided near a distal end of the locking component, and when the locking portion engages with the locked portion, the locking component locks the at least two clamping components.

12. The clip instrument according to claim 11, wherein:
the locking component is a tubular structure, the connecting portion is movably arranged inside the locking component, the locking portion is a locking recess opened inward on a tube wall of the tubular structure, and the locked portion is a locking protrusion facing the tube wall.

13. The clip instrument according to claim 10, wherein:
each of the at least two connecting arms includes an end side portion; when the at least two clamping components are in an open state, movement of the each of the at least two connecting arms from the distal end of the clip instrument toward a proximal end of the clip instrument causes the at least two clamping components to switch from the open state to a closed state, and when the end side portion abuts against the locking component, the at least two clamping components are in the closed state.

14. The clip instrument according to claim 12, wherein:
each of the at least two clamping components includes a first fixing portion, each of the at least two connecting arms includes a second fixing portion, the each of the at least two clamping components and the each of the at least two connecting arms are releasably connected through the first fixing portion and the second fixing portion, the first fixing portion is a protrusion provided on a side wall of the each of the at least two clamping components, and the second fixing portion is a recess provided on the each of the at least two connecting arms.

15. The clip instrument according to claim 14, wherein the locking protrusion constitutes the first fixing portion.

16. The clip instrument according to claim 14, wherein:
each of the at least two clamping components further includes a connecting protrusion, the locking protrusion is provided at a more distal end of the each of the at least two clamping components compared to the connecting protrusion, and the connecting protrusion constitutes the first fixing portion.

17. The clip instrument according to claim 10, wherein:
the clip instrument further includes a connection component, the connection component is slidably connected with the tube body along a first direction and capable of moving relative to the locking component along the first direction.

18. The clip instrument according to claim 17, wherein:
the clamping component includes an abutting portion, movement of the clip from the distal end of the clip instrument toward the proximal end of the clip instrument causes the abutting portion to drive the connection component to move from a first position to a second position, and after the connection component leaves the first position, the locking component locks the at least two clamping components.

19. The clip instrument according to claim 17, wherein:
the locking component includes a connecting channel, and the connection component includes a first limiting shaft;
when the connection component is in a first position, the first limiting shaft is located within the connecting channel; when the connection component is in a second position, the first limiting shaft disengages from the connecting channel.

20. The clip instrument according to claim 10, wherein:
the clamping component includes an elastic portion; when the at least two clamping components are in a closed state, the elastic portions of the at least two clamping components abut against each other.
